(19) United States
(12) Patent Application Publication
(10) Pub. No.: US 2026/0033938 A1
(43) Pub. Date: Feb. 5, 2026

(54) INTERVENTIONAL SYSTEM WITH FILTER

(71) Applicant: ReFlow Medical, Inc., San Clemente, CA (US)

(72) Inventors: Eugene SERINA, Rancho Santa Margarita, CA (US); Jihad Ali MUSTAPHA, Ada, MI (US)

(21) Appl. No.: 18/789,405

(22) Filed: Jul. 30, 2024

(51) Int. Cl. A61F 2/01

(52) U.S. Cl. CPC A61F 2/011

(57) ABSTRACT

Interventional systems described herein can be provided with an interventional device that is positioned for expansion at a target delivery location. The interventional device can act upon a body lumen with a force and/or penetrate a portion of the body lumen. The interventional device can include a filter on a side thereof to capture particles and/or other debris. Such capture can prevent the particles and/or other debris from migrating to other locations downstream of the body lumen.

### TECHNICAL FIELD

The present description relates in general to medical devices, and more particularly to, for example, without limitation, interventional devices with filters.

### BACKGROUND

A variety of devices can be used to treat a body lumen (e.g., vessel) and/or deliver drugs at desired treatment locations within a patient. For example, a stent, such as a drug-eluting stent (DES), can be positioned at the location of a stenosis (arterial narrowing) caused by arteriosclerosis. DESs generally include a drug containing polymer coated over a metal stent or scaffold, or a bioresorbable stent or scaffold composed of a drug-containing polymer. After a DES is delivered to a treatment location within a body lumen (e.g., vessel), it is expanded against a wall of the body lumen (e.g., a vessel wall) and the drug is released via direct contact with the wall. Direct delivery of the drug to the vessel wall enables significantly lower doses than those required via other delivery means (e.g., pills or injections).

### BRIEF DESCRIPTION OF THE DRAWINGS

FIG. 1 shows a partially schematic side view of an example of an interventional system, in accordance with one or more implementations of the present disclosure.

FIG. 2 shows a side view of an example of an interventional device, in accordance with one or more implementations of the present disclosure.

FIG. 3 shows a side view of an interventional device having a filter, in accordance with one or more implementations of the present disclosure.

FIG. 4 shows a side view of a portion of an interventional device, in accordance with one or more implementations of the present disclosure.

FIG. 5 shows a side view of a portion of an interventional device, in accordance with one or more implementations of the present disclosure.

FIG. 6 shows a side view of a portion of an interventional device, in accordance with one or more implementations of the present disclosure.

FIG. 7 shows a side view of a portion of an interventional device in a collapsed state, in accordance with one or more implementations of the present disclosure.

FIG. 8 shows a side view of a portion of the interventional device of FIG. 7 in an expanded state, in accordance with one or more implementations of the present disclosure.

FIG. 9 shows a side view of an interventional system in a delivery state, in accordance with one or more implementations of the present disclosure.

FIG. 10 shows a side view of the interventional system of FIG. 9 in a deployed state, in accordance with one or more implementations of the present disclosure.

FIG. 11 shows a side view of an interventional device having a filter, in accordance with one or more implementations of the present disclosure.

FIG. 12 shows a side view of a portion of an interventional device, in accordance with one or more implementations of the present disclosure.

FIG. 13 shows a side view of a portion of an interventional device, in accordance with one or more implementations of the present disclosure.

FIG. 14 shows a side view of a portion of an interventional device, in accordance with one or more implementations of the present disclosure.

FIG. 15 shows a side view of a portion of an interventional device in a collapsed state, in accordance with one or more implementations of the present disclosure.

FIG. 16 shows a side view of a portion of the interventional device of FIG. 15 in an expanded state, in accordance with one or more implementations of the present disclosure.

FIG. 17 shows a side view of an interventional system in a delivery state, in accordance with one or more implementations of the present disclosure.

FIG. 18 shows a side view of the interventional system of FIG. 17 in a deployed state, in accordance with one or more implementations of the present disclosure.

In one or more implementations, not all of the depicted components in each figure may be required, and one or more implementations may include additional components not shown in a figure. Variations in the arrangement and type of the components may be made without departing from the scope of the subject disclosure. Additional components, different components, or fewer components may be utilized within the scope of the subject disclosure.

### DETAILED DESCRIPTION

The detailed description set forth below is intended as a description of various implementations and is not intended to represent the only implementations in which the subject technology may be practiced. As those skilled in the art would realize, the described implementations may be modified in various different ways, all without departing from the scope of the present disclosure. Accordingly, the drawings and description are to be regarded as illustrative in nature and not restrictive.

The following disclosure describes various embodiments of interventional systems for expandable structures, such as stents or scaffolds, having spikes, flails, or other protruding features for penetrating target tissue and/or delivering drugs within a human patient, and associated devices and methods. The interventional systems can be configured to deliver and position expandable structures within a body lumen (e.g., vessel). In addition, these interventional systems can also be configured to deploy and expand the expandable structures in the body lumen. The interventional systems can further be configured to engage with the expanded structure and collapse the structure for removal from the body lumen. A filter can be provided with the interventional device to capture debris therein while allowing selective perfusion of fluids there through. The filter can be coupled to a frame of the interventional device to provide secure an consistent placement thereof. Such interventional systems are expected to simplify and expedite transluminal procedures to more effectively deliver and position expandable structures within target tissues. The interventional systems can be used with more than one procedure, such as deployment of an expandable structure, when configured to re-capture the deployed expandable structure.

In particular, interventional systems described herein can be provided with an interventional device that is positioned for expansion at a target delivery location. The interventional device can act upon a body lumen with a force and/or penetrate a portion of the body lumen. The interventional device can include a filter on a proximal side thereof to capture particles and/or other debris. Such capture can prevent the particles and/or other debris from migrating to other locations downstream of the body lumen.

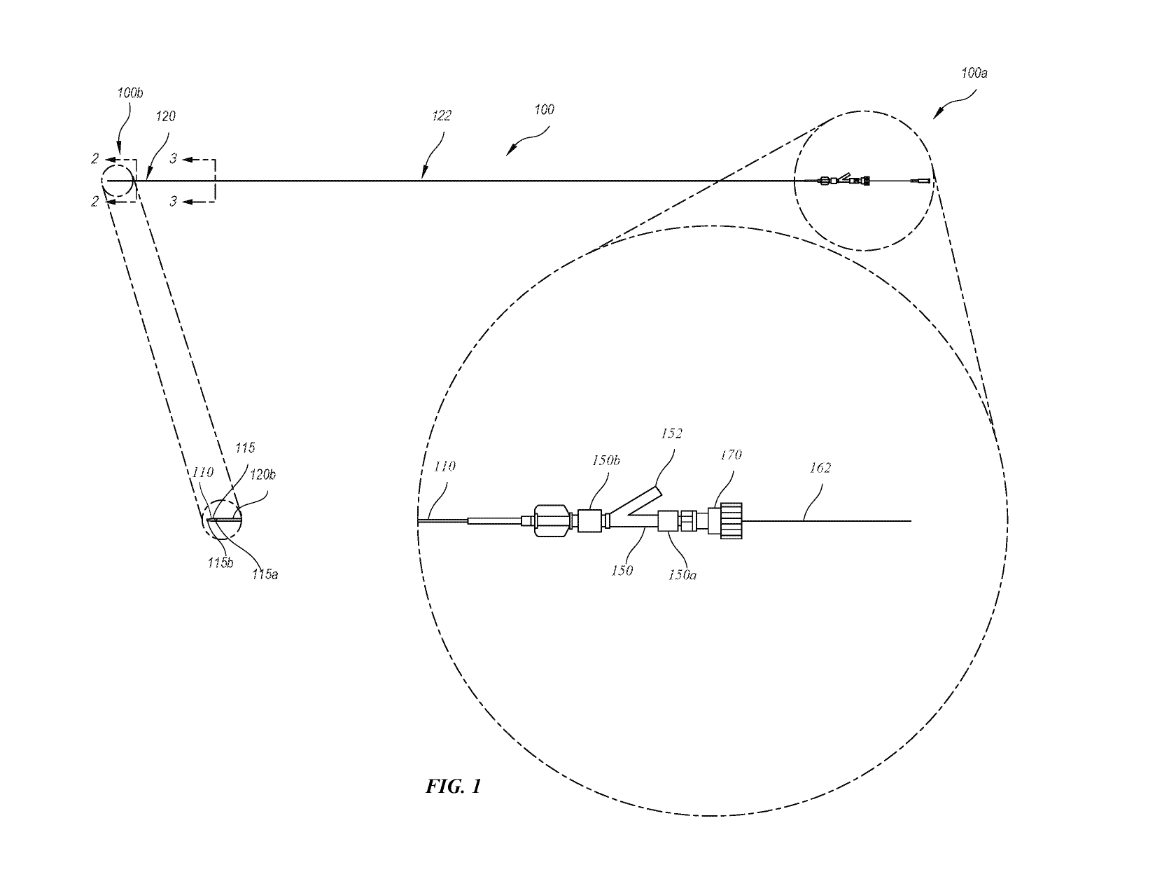

Certain details are set forth in the following description and FIGS. 1-18 to provide a thorough understanding of various embodiments of the disclosure. To avoid unnecessarily obscuring the description of the various embodiments of the disclosure, other details describing well-known structures and systems often associated with expandable structures, protruding features, and the components or devices associated with the manufacture of such structures are not set forth below. Moreover, many of the details and features shown in the figures are merely illustrative of particular embodiments of the disclosure. Accordingly, other embodiments can have other details and features without departing from the spirit and scope of the present disclosure. A person of ordinary skill in the relevant art will therefore understand that the present technology, which includes associated devices, systems, and procedures, may include other embodiments with additional elements or steps, and/or may include other embodiments without several of the features or steps shown and described below with reference to FIGS. 1-18. Furthermore, various embodiments of the disclosure can include structures other than those illustrated in the figures and are expressly not limited to the structures shown in the figures.

FIG. 1 shows a partially schematic side view of interventional system 100 for an interventional device in a delivery state (e.g., low-profile or collapsed state). Interventional system 100 includes outer shaft 120 (e.g., a catheter) having one or more lumens for containing one or more elongate members (e.g., inner shaft 110 and/or guidewire 162). In some embodiments, outer shaft 120 may also include one or more layers. In these embodiments, for example, the layers of outer shaft 120 can include an inner layer, an outer layer, a liner, or a combination thereof. Each of the layers can be formed from materials including a polymer, high-density polyethylene (HDPE), polytetrafluoroethylene, silicone, Pebax® (polyether block amide) or a combination thereof. In some embodiments, each of the layers of outer shaft 120 are formed from the same material. In other embodiments, however, one or more of the layers may be formed from different materials.

Inner shaft 110 can extend from connector 150, through outer shaft 120, and beyond distal portion 120b of outer shaft 120. Inner shaft 110 can be formed as a tubular structure (with or without a slit), such as a coiled tube, a braided tube, a reinforced tube, or a combination thereof, and may be constructed of a polymer material, such as a polyimide. Interventional system 100 can include a guidewire within inner shaft 110 and accessible at a proximal end of interventional system 100.

In the detailed view of the distal portion 100b of interventional system 100, tip 115 (e.g., an atraumatic tip) is disposed on a distal terminal end of inner shaft 110. As illustrated, tip 115 is adjacent to a distal terminal end of outer shaft 120. At least a portion of tip 115 can have the same cross-sectional dimension as outer shaft 120, or tip 115 may have a different cross-sectional dimension. In some embodiments, distal end 115b of tip 115 is tapered such that distal end 115b has a smaller cross-sectional dimension compared to proximal end 115a of tip 115. Distal and/or proximal edges of tip 115 may be curved/rounded so as to prevent tip 115 from getting caught (e.g., stuck) on other portions of interventional system 100 during delivery, positioning, deployment, etc. Tip 115 can be formed of the same material(s) as outer shaft 120. In other embodiments, however, tip 115 can be formed from different material(s) than outer shaft 120.

Inner shaft 110 and outer shaft 120 can be sized and shaped for intravascularly accessing a target site (e.g., treatment site) of the patient. In some embodiments, for example, the outer shaft 110 has a length of about 150 cm to about 180 cm and a suitable cross-sectional dimension for positioning within a subject’s vasculature. The length of inner shaft 110 can be a working length, such as a length that can be positioned within a subject’s vasculature. In some embodiments, for example, the working length is about 70 cm to about 300 cm, about 150 cm to about 250 cm, or about 70 cm, about 80 cm, about 90 cm, about 100 cm, about 110 cm, about 120 cm, about 130 cm, about 140 cm, about 150 cm, about 160 cm, about 170 cm, about 180 cm, about 190 cm, about 200 cm, about 210 cm, about 220 cm, about 230 cm, about 240 cm, about 250 cm, about 260 cm, about 270 cm, about 280 cm, about 290 cm, or about 300 cm. In other embodiments, outer shaft 120 has a length of about 130 centimeters (cm) to about 140 cm and a cross-sectional dimension of about 4 French, about 5 French, or about 6 French. The length of outer shaft 120 can be a working length, such as a length that can be positioned within a subject’s vasculature. In some embodiments, the working length is about 50 cm to about 200 cm, about 100 cm to about 150 cm, or about 50 cm, about 60 cm, about 70 cm, about 80 cm, about 90 cm, about 100 cm, about 110 cm, about 120 cm, about 125 cm, about 130 cm, about 135 cm, about 140 cm, about 145 cm, about 150 cm, about 155 cm, about 160 cm, about 170 cm, about 180 cm, about 190 cm, or about 200 cm.

In the detailed view of proximal portion 100a of interventional system 100 in FIG. 1, proximal end 120c of the outer shaft is coupled to outer shaft hub 140. In the illustrated embodiment, outer shaft hub 140 is coupled to outer shaft 120 (e.g., via bonding). In other embodiments, however, proximal end 120c of the outer shaft is directly coupled to outer shaft hub 140.

Outer shaft hub 140 is further coupled to connector 150 (e.g., y-connector) having a lumen extending therethrough (not shown). In particular, distal end 150b of connector 150 can be coupled to outer shaft hub 140 via a mating feature and a receiving feature (not shown). The mating and receiving features can be coupled to the proximal portion of outer shaft 120 or distal end 150b of connector 150. Connector 150 further includes port 152 extending radially and/or longitudinally therefrom. Interventional system 100 can optionally include hemostasis connector 170 coupled to proximal end 150a of connector 150. While proximal end 120c is illustrated with particular components in a particular arrangement, it will be understood that additional or fewer components can be included in similar or other arrangements to meet the needs of the system.

Interventional system 100 is configured to carry an interventional device, discussed further herein, in a delivery/collapsed state within a distal portion of outer shaft 120. The interventional device can be at least partially ensheathed by outer shaft 120. In some embodiments, the interventional device can be fixedly or removably coupled to inner shaft 110. Although interventional system 100 is illustrated as a delivery system for interventional devices, such as stents, it will be appreciated that embodiments of the present technology can also include cages, meshes, balloons, membranes, tubular structures, circumferential bodies, expandable elements, expandable membranes, expandable structures, expandable tubular structures, and circumferentially expandable catheter tips with and without guidewire lumens.

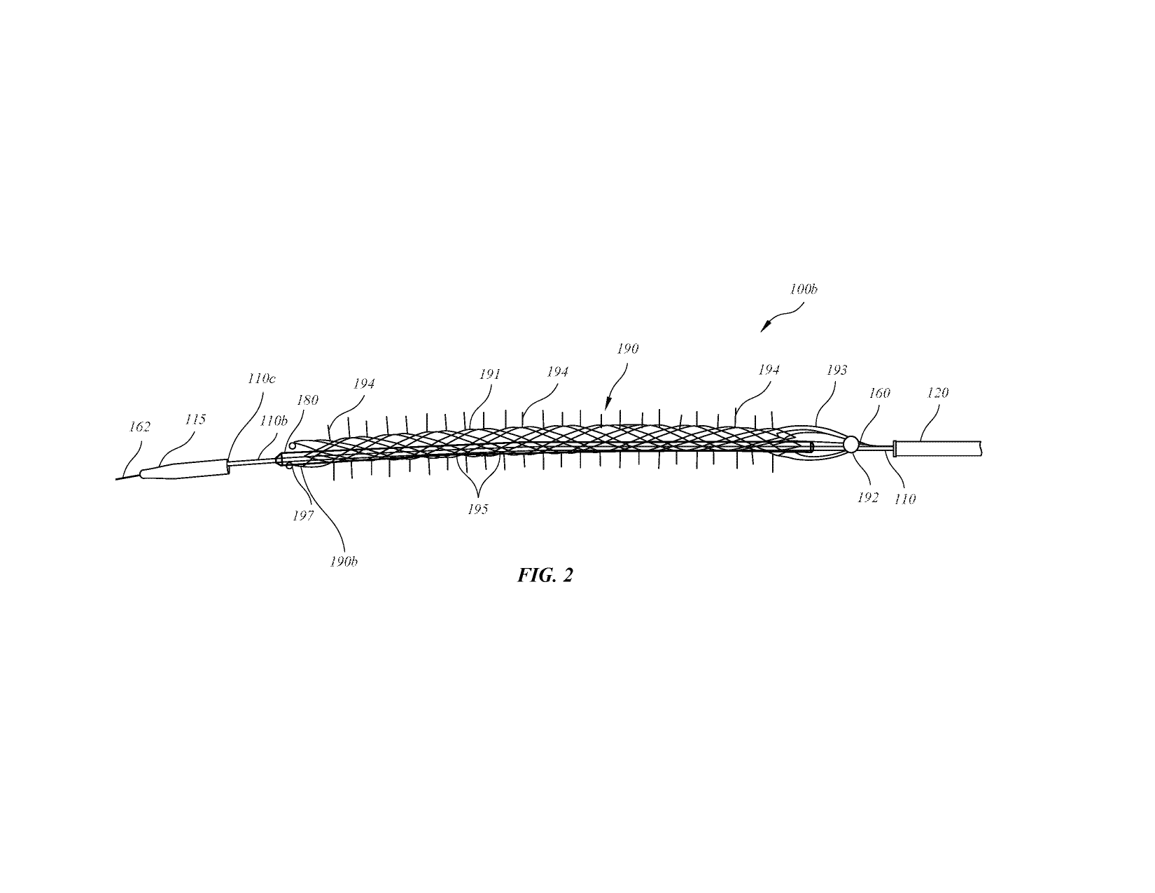

FIG. 2 shows a side view of distal portion 100b of interventional system 100 of FIG. 1 in a deployed state. In the illustrated embodiment, interventional device 190 extends over balloon 180 and is coupled to the delivery catheter shaft and has been unsheathed from distal portion 120b of outer shaft 120. Proximal visualization marker 192 is disposed on one or more elongate members (e.g., stabilizing wire 160) near a proximal portion of interventional device 190 and distal visualization markers 197 are disposed on distal portion 190b of interventional device 190. In some embodiments, proximal visualization marker 192 and/or distal visualization marker 197 may be disposed on stabilizing wire 160. Visualization markers 192 and/or 197 can be formed from any material that can be visualized while interventional device 190 is intravascularly positioned (e.g., within a target blood vessel). In one embodiment, for example, visualization markers 192 and/or 197 are radiopaque markers. Different elongate members can be moveable relative to each other or secured to each other (e.g., to move in unison). For example, stabilizing wire 160 can be connected to inner shaft 110, such that movement of inner shaft 110 correspondingly urges interventional device 190 with stabilizing wire 160. Alternatively, stabilizing wire 160 can be independently movable relative to inner shaft 110.

Tip 115 is disposed on terminal end 110c of inner shaft 110 and can surround terminal end 110c extending proximally along distal portion 110b and/or distally from terminal end 110c. Inner shaft 110 extends distally from distal portion 120b of outer shaft 120, through a lumen of interventional device 190, and, optionally, extends distally from the distal end of interventional device 190. In the deployed state, protruding features 194 extend radially from a longitudinal axis of interventional device 190.

Inner shaft 110 can also include inflatable balloon 180. Inflatable balloon 180 can be axially overlapping with interventional device 190, distal to interventional device 190, or proximal to interventional device 190 while interventional device 190 is in a delivery state (e.g., low-profile or collapsed state) within outer shaft 120 and/or while interventional device 190 is initially deployed from the delivery state.

Guidewire 162 can extend through inner shaft 110 and beyond tip 115. Accordingly, guidewire 162 can be advanced ahead of other portions of interventional system 100. Inner shaft 110, interventional device 190, and outer shaft 120 can be advanced over guidewire 162 until interventional device 190 is aligned with a desired target delivery location. The length of guidewire 162 that overlaps other portions of interventional system 100 can be within inner shaft 110, so that it does not interfere with any other components of interventional system 100.

As further shown in FIG. 2, interventional device 190 is provided with frame 191 and multiple protruding features 194. Frame 191 and protruding features 194 can be configured to radially expand after interventional device 190 has been unsheathed from outer shaft 120. Interventional device 190 can be self-expanding upon release from a constraint. Additionally or alternatively, interventional device 190 can be expandable by radial forces applied from balloon 180 that is inflated while within interventional device 190. Frame 191 can include multiple struts 195 arranged in a pattern that supports compression, expansion, flexibility, and bendability of interventional device 190. Frame 191 can form a generally cylindrical shape along at least a portion of interventional device 190. At least a portion of each protruding feature 194 can extend at least partially distally from frame 191 (e.g., towards distal portion 190b). For example, at least a portion of each protruding feature 194 can extend parallel to a longitudinal axis of interventional device 190. At least a portion (e.g., terminal end portion) of each protruding feature 194 can extend at least partially radially away from frame 191. For example, at least a portion of each protruding feature 194 can extend radially outwardly (e.g., perpendicular to) the longitudinal axis of interventional device 190. With at least a portion of each protruding feature 194 extending distally from frame 191, protruding features 194 can be readily retracted into outer shaft 120 by folding down and extending distally when outer shaft 120 is advanced from a proximal side of interventional device 190 in a distal direction over interventional device 190. Protruding features 194 can optionally include drugs for delivery to a target delivery location upon expansion of interventional device 190. However, it will be understood that interventional device 190 can omit drugs for delivery and treat a target delivery location by penetrating tissue with protruding features 194.

Frame 191, struts 195, and/or protruding features 194 can be composed of or formed from a variety materials including, e.g., nitinol, cobalt chromium, stainless steel, any of a variety of other metals or metal alloys, or a combination thereof. Frame 191, struts 195, and/or protruding features 194 may also be composed of or formed from bioresorbable biodegradable, nanoporous or non-bioresorbable, non-biodegradable, non-nanopourous materials including, e.g., one or more polymers, nitinol, plastic materials, etc., or a combination thereof. In some embodiments, frame 191 and struts 195 can be formed from a bioresorbable material and protruding features 194 can be formed from a non-bioresorbable material, such as nitinol. In these embodiments, protruding features 194 can remain engaged with or penetrating a portion of the body lumen after frame 191 and struts 195 bio-resorb. After frame 191 and struts 195 bio-resorb, the body lumen where interventional device 190 had been expanded is no longer partially occluded by frame 191 and struts 195 allowing for larger volumes of fluids, such as aqueous pharmaceutical compositions, to pass through the body lumen and contact the luminal wall. Protruding features 194 may also be formed of a bio-resorbable material and, once interventional device 190 has bio-resorbed, the spaces in the body lumen wall vacated by protruding features 194 can be contacted by the fluids passing through the body lumen. In this way, interventional device 190 can increase a surface area of the body lumen wall contacted by the fluid.

Protruding features 194 may also be carried by more than one strut 195, frame 191, or a combination thereof. Protruding features 194 may be integrally formed with struts 195, for example by bending or twisting a portion of one or more struts and/or frame 191 away from a longitudinal axis of interventional device 190 or, alternatively, protruding features 194 may be separate, discrete components that are attached to desired locations along struts 195 and/or frame 191.

Frame 191 of interventional device 190 can be connected to stabilizing wire 160 and/or inner shaft 110 by intermediate portion 193. Intermediate portion 193 can include multiple struts that may have varying widths to aide in column strength for deploying and retraction that extend from different portions of frame 191, for example connecting to different circumferential portions at an end of frame 191. Struts 195 of intermediate portion 193 can extend to the same or different axial locations along stabilizing wire 160 and/or inner shaft 110. The arrangement of struts 195 of intermediate portion 193 can maintain an open central space along the entire length of interventional device 190.

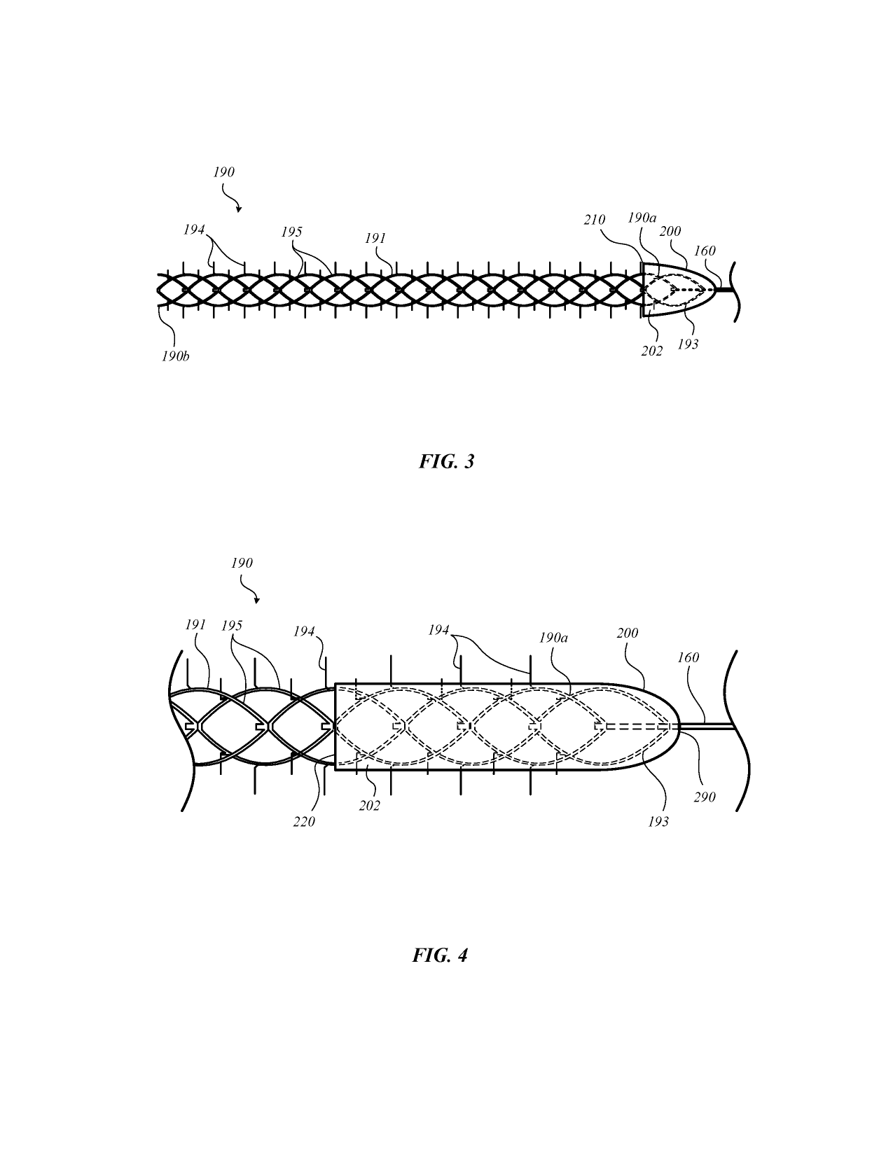

Referring now to FIGS. 3–10, an interventional device can include a filter covering a proximal portion of the frame . As shown in FIG. 3, filter 200 can be provided at proximal portion 190a of frame 191. For example, filter 200 can extend circumferentially and entirely about proximal portion 190a of frame 191, which can optionally include intermediate portion 193 of frame 191. Filter 200 can extend from stabilizing wire 160 and distally toward distal portion 190b of frame 191. Filter 200 can have a shape that generally conforms to and/or matches that of frame 191 at portions covered thereby. In some embodiments, filter 200 can have a maximum outer dimension that is greater than a maximum outer dimension of frame 191. As interventional device 190 expands, filter 200 can be urged radially outwardly along with radial expansion of frame 191. In some embodiments, filter 200 can constrain radial expansion of frame 191, for example where a maximum outer dimension of filter 200 is equal to or less than a maximum outer dimension of frame 191. Interventional device 190 of FIG. 3 can be implemented in any one of interventional systems 100 of FIGS. 1 and 2.

It will be understood that filter 200 can cover any portion and/or length of frame 191. In some embodiments, filter 200 covers at least proximal portion 190a, distal portion 190b, and/or intermediate portion 193 of frame 191. For example, filter 200 can extend at least to a portion of frame 191 that defines a maximum outer dimension of frame 191. By further example, filter 200 can extend over about 10%, about 20%, about 30%, about 40%, about 50%, about 60%, about 70%, about 80%, about 90%, or about 100% of the total length of frame 191. By further example, filter 200 can extend over about 0.25 mm, about 0.5 mm, about 1 mm, about 2 mm, about 3 mm, about 4 mm, about 5 mm, about 6 mm, about 7 mm, about 8 mm, about 9 mm, about 10 mm, about 12 mm, about 14 mm, about 16 mm, about 18 mm, about 20 mm, about 30 mm, about 40 mm, about 50 mm, about 60 mm, about 70 mm, about 80 mm, about 90 mm, or about 100 mm along the length of frame 191. In some embodiments, filter 200 covers an entire length of frame 191.

Filter 200 can include wall 202 comprising a material forming a sheet or film having a surface. Wall 202 of filter 200 can include a mesh and/or other porous structure. Wall 202 may be continuous and uninterrupted (e.g., without one or more discontinuities) about a circumference thereof. In some embodiments, the mesh is selectively permeable. For example, wall 202 may be textured, cut, or perforated to provide selective perfusion of fluid and/or materials through wall 202 of filter 200 (e.g., from within filter 200 to outside or beyond filter 200). In some embodiments, wall 202 of filter 200 allows diffusion and/or other passage of a fluid there through while preventing passage of larger debris, such as a thrombus, embolus, or other materials encountered in a body lumen and captured within filter 200. In some embodiments, wall 202 of filter 200 includes a braided, woven, knitted, and/or laminated material. The pores of such a mesh and/or other openings can be formed as interstices between portions (e.g., strands) of materials and/or cut (e.g., laser cut) openings. In some embodiments, pores of the mesh have a maximum, minimum, and/or average dimension of 5–200 microns, 40–150 microns, and/or 50–100 microns. In some embodiments, wall 202 of filter 200 can include pores extending there through. For example, a laser cutting procedure may be applied to provide pores of a selected size, quantity, and distribution to wall 202 of filter 200. By further example, wall 202 of filter 200 can be formed in a manner that produces pores of a selected size, quantity, and distribution. The pores can have a diameter that is less than about 5 microns, about 5 microns, about 10 microns, about 15 microns, about 20 microns, about 25 microns, about 30 microns, about 35 microns, about 40 microns, about 45 microns, about 50 microns, about 100 microns, about 150 microns, or more than about 150 microns.

Wall 202 of filter 200 may be produced of a polymer material. For example, wall 202, or a portion thereof, may be of polytetrafluoroethylene (“PTFE”), expanded polytetrafluoroethylene (“ePTFE”), fluorinated ethylene propylene (“FEP”), low density polyethylene (“LDPE”), polypropylene, polyvinyl chloride (“PVC”), polydimethylsiloxane, polyethylene terephthalate (“PET”) (Dacron®), polyurethane, polyamides (nylon), polyether urethane, polycarbonate, polysulfones, polymethyl methacrylate, poly 2-hydroxy-ethylmethacrilate (PHEMA), or combinations thereof. The polymer may provide flexibility and be readily conformable when manipulated during delivery and deployment. The polymer may also provide a coefficient of friction that allows components covered by the polymer to smoothly move past structures, such as a catheter, that constrains the components. The material for filter 200, a portion of filter 200, and/or wall 202 may be unsintered (e.g. an unsintered polymer such as unsintered PTFE or ePTFE). According to one or more implementations, filter 200 can be unsintered except where portions (e.g., layers) thereof are joined, adhered, or welded to each other. The term “unsintered” may include a property of a material that has not been subject to heat of a sintering process. An unsintered material may be a powder that has been preformed under pressure. A sintered material is one that has been exposed to heat to change the cross-linking structure of the material.

In some embodiments, as further shown in FIG. 3, filter 200 transitions from a first cross-sectional dimension at frame 191 to a second cross-sectional dimension at stabilizing wire 160, wherein the first cross-sectional dimension is larger than the second cross-sectional dimension. For example, filter 200 can form open end 210, which can define a distalmost extent of filter 200. Open end 210can further define the first cross-sectional dimension, which can be a maximum cross-sectional dimension of filter 200. For example, an inner surface of wall 202 of filter 200 can abut and/or be close to an outer periphery of frame 191. In some embodiments, an inner dimension of wall 202 of filter 200 is equal to or approximately equal to an outer dimension of frame 191. In some embodiments, wall 202 of filter 200 constrains frame 191 while interventional device 190 is in an expanded state. From the first (e.g., maximum) cross-sectional dimension, filter 200 tapers to the second cross-sectional dimension along with a tapering dimension of frame 191, such as along intermediate portion 193. Stabilizing wire 160 can extend through filter 200, while filter 200 provides limited openings for passage therethrough in a vicinity of stabilizing wire 160. Accordingly, filter 200 can define an opening at open end 210 for receiving debris therein, and filter 200 can further capture such debris to be surrounded by wall 202 and blocked by a closure formed at a portion of filter 200 at stabilizing wire 160.

Referring now to FIG. 4, a filter can be provided in an engaged arrangement with respect to a frame of an interventional device with one or more protruding features. As shown in FIG. 4, filter 200 can extend across a portion of frame 191 that supports one or more protruding features 194. In some embodiments, as protruding features 194 extend radially outwardly from frame 191 and as filter 200 is provided in close proximity to frame 191, one or more protruding features 194 positioned along the length of filter 200 extends through and beyond an outer surface of filter 200. In some embodiments, one or more protruding features 194 transition from a constrained state within filter 200 while interventional device 190 is in a collapsed state to an extended state protruding through wall 202 of filter 200 while interventional device 190 is in an expanded state. In some embodiments, while one or more protruding features 194 extend through wall 202 of filter 200, at least a portion of filter 200 is axially secured to frame 191. For example, filter 200 can be engaged by one or more protruding features 194 extending therethrough, and filter 200 can be maintained in a given axial alignment with respect to frame 191, such that at least a portion of filter 200 does not move axially with respect to frame 191. It will be understood that while some portions (e.g., a distal portion) of filter 200 can be secured, other portions (e.g., a proximal portion) of filter 200 can be permitted to move, such as portions not engaged by and/or between one or more protruding features 194. In some embodiments, one or more protruding features 194 of interventional device 190 are positioned to be axially offset with respect to filter 200. For example, at least a portion (e.g., proximal portion 190a) of frame 191 can be covered by filter 200, at least a portion (e.g., distal portion 190b) of frame 191 can be uncovered by filter 200, and one or more protruding features 194 of interventional device 190 can extend radially outwardly without extending through filter 200. In some embodiments, every one of protruding features 194 of interventional device 190 is positioned to extend through filter 200. Interventional device 190 of FIG. 4 can be implemented in any one of interventional systems 100 of FIGS. 1 and 2 and/or configured to include one or more of the features described herein with respect to interventional device 190 of FIG. 3.

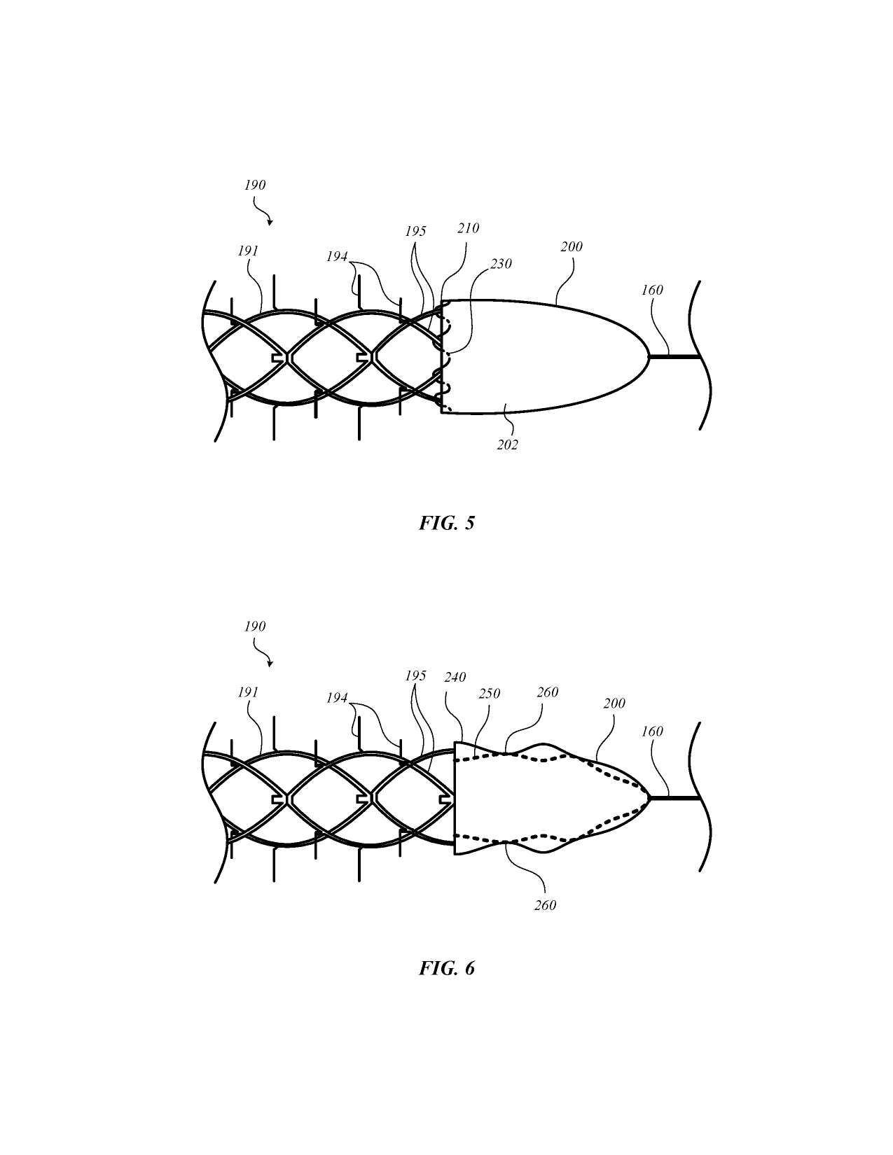

Referring now to FIG. 5, a filter can be provided in an engaged arrangement with respect to a frame of an interventional device with one or more strands. As shown in FIG. 5, filter 200 can extend across a portion of frame 191 that defines struts 195. In some embodiments, open end 210 of filter 200 is secured to frame 191 by one or more strands 230 that extend through at least a portion of wall 202 and about one or more of struts 195. For example, strand 230 can extend in an alternating pattern to extend through the wall 202 (e.g., at or near open end 210) and about an inner side of struts 195. Accordingly, strand 230 can provide tension that pulls filter 200 toward frame 191. In some embodiments, strand 230 extends about an entire circumference of frame 191 and/or open end 210. Strand 230 can optionally extend to an inner side of each strut 195 across which it extends. Between each strut 195, strand 230 can extend through and/or otherwise engage wall 202 of filter 200 at or near open end 210. In some embodiments, while strand 230 extends through wall 202 of filter 200 and about one or more struts 195, at least a portion (e.g., open end 210) of filter 200 is axially and/or radially secured to frame 191. For example, filter 200 can be engaged by one or more strands 230 extending therethrough, and filter 200 can be maintained in a given axial alignment with respect to frame 191, such that at least a portion (e.g., open end 210) of filter 200 does not move axially with respect to frame 191. It will be understood that while some portions (e.g., open end 210 and/or distal portion) of filter 200 can be secured, other portions (e.g., a proximal portion) of filter 200 can be permitted to move, such as portions not engaged by and/or between one or more strands 230. In some embodiments, at least a portion (e.g., proximal portion 190a) of frame 191 can be covered by filter 200, at least a portion (e.g., distal portion 190b) of frame 191 can be uncovered by filter 200, and one or more strands 230 can secure open end 210 there between. In some embodiments, one or more strands 230 secure open end 210 of filter 200 to a distal end of frame 191. Interventional device 190 of FIG. 5 can be implemented in any one of interventional systems 100 of FIGS. 1 and 2 and/or configured to include one or more of the features described herein with respect to interventional devices 190 of FIGS. 3 and 4. For example, open end 210 of filter 200 can be coupled to frame 191 by one or more strands 230 and/or one or more protruding features 194 of interventional device 190 can extend through wall 202 of filter 200.

Referring now to FIG. 6, a filter can be provided in an engaged arrangement with respect to a frame of an interventional device with coupled layers. As shown in FIG. 6, filter 200 can extend across a portion of frame 191 that defines struts 195. In some embodiments, filter 200 includes multiple layers, such as outer layer 240 and inner layer 250. In some embodiments, at least a portion of outer layer 240 extends across a radially outer surface of frame 191 (e.g., radially outer surfaces of struts 195), and at least a portion of inner layer 250 extends across a radially inner surface of frame 191 (e.g., radially inner surfaces of struts 195. For example, strand 230 can extend in an alternating pattern to extend through the wall 202 (e.g., at or near open end 210) and about an inner side of struts 195. In some embodiments, outer layer 240 and inner layer 250 are coupled to each other at one or more locations along a length of frame 191. For example, one or more couplings 260 can be provided at one or more interstices defined by struts 195. Such interstices can define openings that extend radially through frame 191. At such locations, outer layer 240 and inner layer 250 can be joined together by one or more couplings 260. In some embodiments, couplings 260 are formed by adhesion, fusion, and/or sintering of outer layer 240 and inner layer 250. In some embodiments, couplings 260 are formed by stitching, interweaving, and/or braiding of outer layer 240 and inner layer 250. Couplings 260 can be provided at various locations, including circumferentially and/or axially distributed across filter 200 and/or frame 191. While outer layer 240 and inner layer 250 can extend across radially opposite sides of corresponding struts 195, outer layer 240 and inner layer 250 can be joined together by couplings 260. As such, the locations of such couplings 260 with respect to frame 191 can be maintained to secure filter 200 to frame 191. In some embodiments, couplings 260 maintain at least a portion of filter 200 to be axially and/or radially secured to frame 191, such that at least a portion (e.g., distal portion) of filter 200 does not move axially with respect to frame 191. It will be understood that while some portions (e.g., distal portion) of filter 200 can be secured, other portions (e.g., a proximal portion) of filter 200 can be permitted to move, such as portions not engaged by couplings 260 between struts 195. In some embodiments, at least a portion (e.g., proximal portion 190a) of frame 191 can be covered by filter 200, at least a portion (e.g., distal portion 190b) of frame 191 can be uncovered by filter 200. Interventional device 190 of FIG. 6 can be implemented in any one of interventional systems 100 of FIGS. 1 and 2 and/or configured to include one or more of the features described herein with respect to interventional devices 190 of FIGS. 3–5. For example, outer and inner layers 240 and 250 of filter 200 can be coupled together between some of struts 195 of frame 191, open end 210 of filter 200 can be coupled to frame 191 by one or more strands 230, and/or one or more protruding features 194 of interventional device 190 can extend through wall 202 (e.g., outer and inner layers 240 and 250) of filter 200.

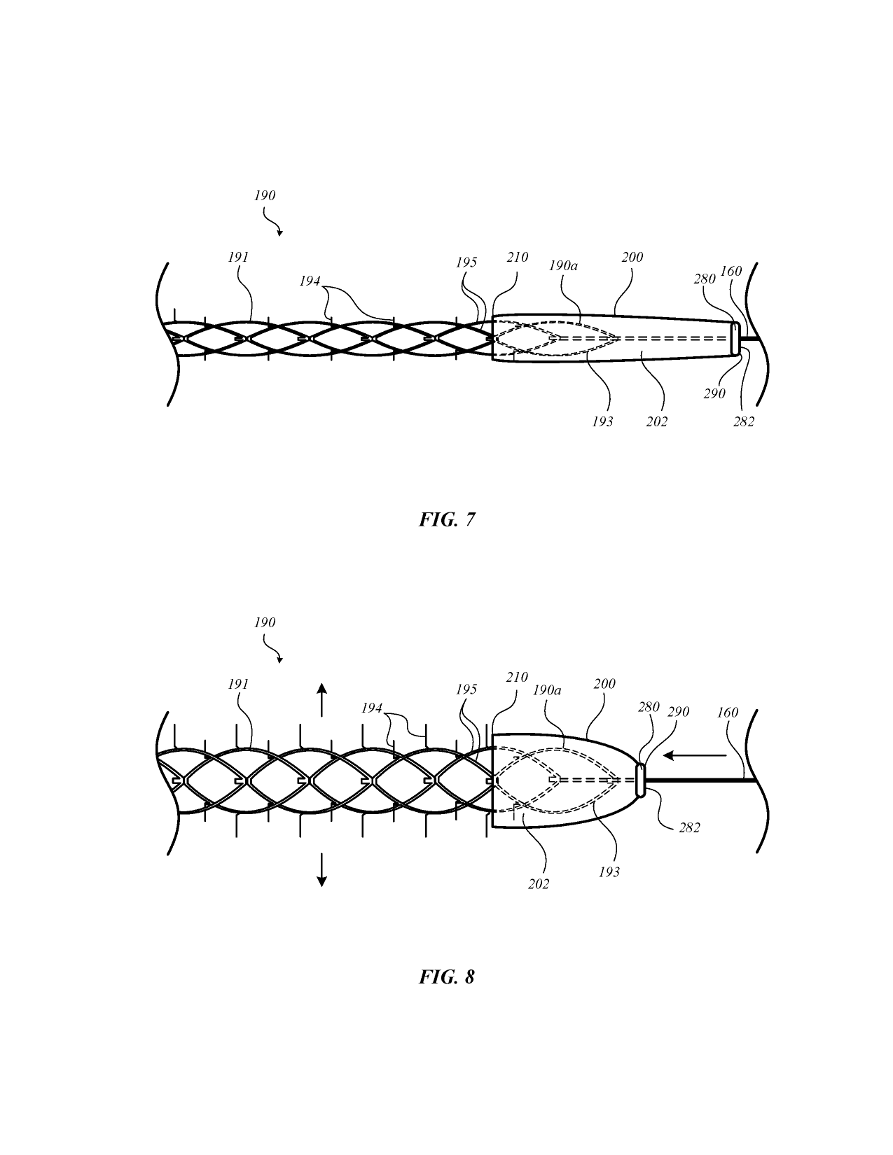

Referring now to FIGS. 7 and 8, an interventional device can be provided with features to facilitate expansion of a frame thereof with a filter thereon. Interventional device 190 of FIGS. 7 and 8 can be implemented in any one of interventional systems 100 of FIGS. 1 and 2 and/or configured to include one or more of the features described herein with respect to interventional devices 190 of FIGS. 3–6. For example, engagement between filter 200 and frame 191 can be provided with one or more protruding features 194 extending from frame 191 and through filter 200, open end 210 of filter 200 can be coupled to frame 191 by one or more strands, and/or outer and inner layers of filter 200 can be coupled together between some of struts 195 of frame 191. It will be understood that interventional device 190 can undergo changes to its size and/or shape when transitioning between a collapsed state (FIG. 7) and an expanded state (FIG. 8). Where filter 200 is coupled to frame 191 at or near open end 210 thereof, tapered end 290 of filter 200 can be configured to accommodate such changes while maintaining engagement at or near open end 210. In some embodiments, filter 200 includes collar 280 at or near tapered end 290 of filter 200. Collar 280 can provide opening 282 through which stabilizing wire 160 and/or another component can extend. Opening 282 can be configured (e.g., sized) to accommodate passage of stabilizing wire 160 and/or another component while also limiting passage of debris therethrough. In some embodiments, collar 280 can include a material and/or construction that is different from that of wall 202 of filter 200. For example, collar 280 can include an annular ring of a flexible material, such as a plastic, a polymer (e.g., silicon), and/or a combination thereof. Collar 280 can facilitate sliding along stabilizing wire 160. For example, while open end 210 or another distal portion of filter 200 is secured to frame 191, tapered end 290 of filter 200 can be permitted to move (e.g. slide) to accommodate expansion of frame 191. In some embodiments, as shown in FIG. 8, frame 191 expands radially outwardly. Radial expansion of frame 191 can optionally include longitudinal foreshortening of frame 191. In some embodiments, as further shown in FIG. 8, radial expansion of frame 191 can also urge filter 200 to expand radially outwardly and/or foreshorten longitudinally. During expansion of frame 191, collar 280 can travel longitudinally (e.g., toward frame 191) to accommodate such transitions. Longitudinal travel of collar 280 in a distal direction can optionally be limited by contact with frame 191 (e.g., at intermediate portion 193) and/or another structure. In some embodiments, interventional device 190 can transition from the expanded state of FIG. 8 to the collapsed state of FIG. 7. Such a transition can include longitudinal motion (e.g., sliding) of collar 280 away from frame 191 (e.g., proximally).

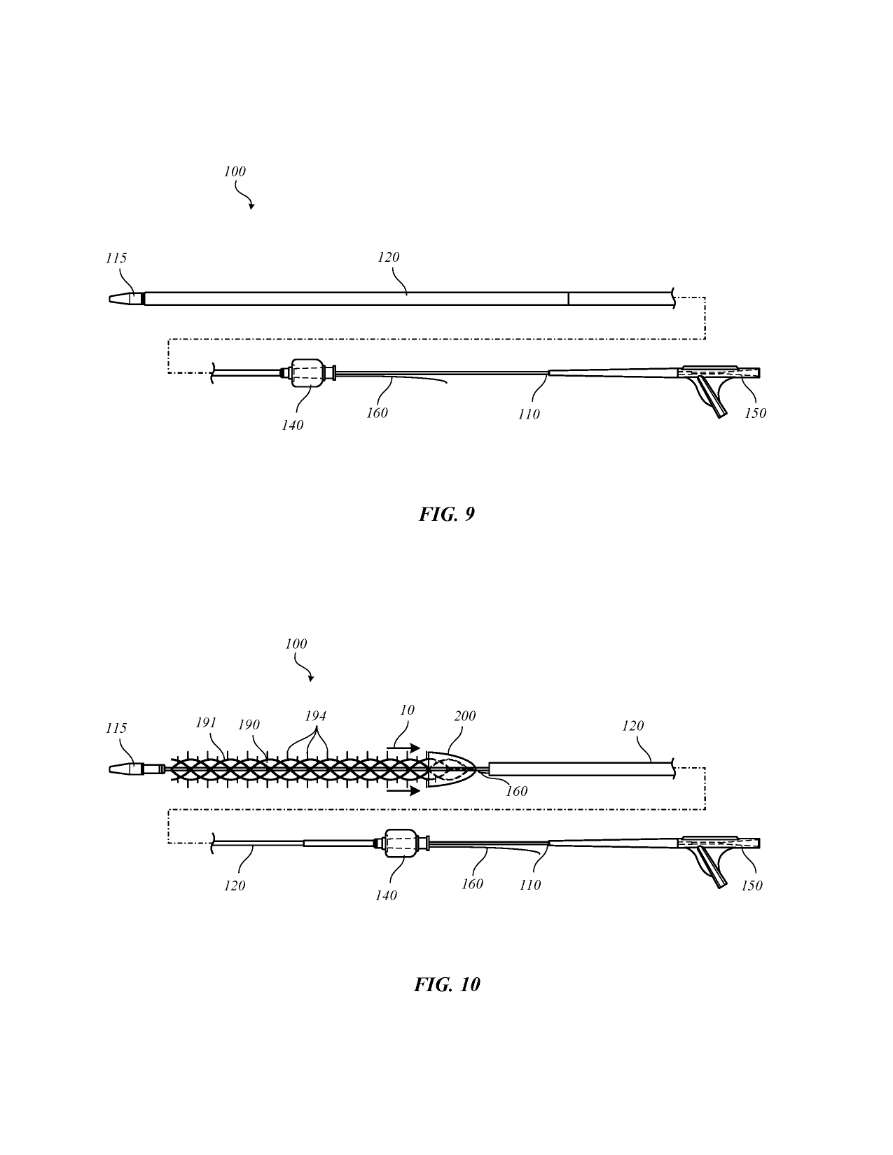

Referring now to FIGS. 9 and 10, an interventional device can be delivered to a target location for deployment thereat. Methods described herein provide delivery of interventional device 190 to a target delivery location by operation of interventional system 100. While methods in their various stages are discussed and illustrated herein, it will be understood that multiple variations of each method are also contemplated. For example, the methods can be performed in various orders of operations, with additional operations, or with fewer operations. Interventional system 100 of FIGS. 9 and 10 can be implemented as any one of interventional systems 100 of FIGS. 1 and 2, and interventional devices 190 of FIGS. 9 and 10 can be configured to include one or more of the features described herein with respect to interventional devices 190 of FIGS. 3-8.

As shown in FIG. 9, interventional system 100 is provided with outer shaft 120 covering or ensheathing other components of interventional system 100. For example, outer shaft 120 can extend to tip 115 positioned at a distal end of inner shaft 110. Inner shaft 110 can extend within outer shaft 120, with a length thereof accessible proximal to a proximal end of outer shaft 120 (e.g., at outer shaft hub 140). Additionally or alternatively, connector 150 can be accessible proximal to a proximal end of outer shaft 120 (e.g., at outer shaft hub 140). As discussed above, a guidewire can be advanced ahead of tip 115 (e.g., through inner shaft 110) to provide a pathway for advancement of other components of interventional system 100. In some embodiments, stabilization wire 160 is accessible on a proximal side of interventional system 100, such through outer shaft hub 140. For example, stabilization wire 160 can be connected to an interventional device within outer shaft 120.

As shown in FIG. 10, interventional system 100 can be provided with interventional device 190 that is positioned over inner shaft 110 (and/or an inflatable balloon, not shown in FIG. 10) for expansion and delivery of interventional device 190 to a target delivery location. By positioning interventional device 190 over and about inner shaft 110, interventional device 190 can be ready to be expanded immediately upon unsheathing with respect to outer shaft 120.

As further shown in FIG. 10, outer shaft 120 can be moved to unsheath interventional device 190 and other components of interventional system 100. For example, once the distal region of interventional system 100 is positioned at a desired location, outer shaft 120 is configured to be at least partially proximally retracted relative to inner shaft 110 by retracting outer shaft hub 140 relative to connector 150. Once outer shaft 120 is partially retracted, at least a portion of interventional device 190 and/or a balloon (where provided) is unsheathed. In some embodiments, frame 191, protruding features 194, and/or filter 200 of interventional device 190 are configured to radially expand outwardly away from inner shaft 110.

As used herein, movement of various components can be relative to other components of interventional system 100 and/or relative to a position apart from interventional system 100 (e.g., a position within the anatomy of the patient, target delivery location, and/or tissue). The directions “proximal” and “distal” can be with respect to interventional system 100, a component thereof, and/or a position apart from interventional system 100. For example, movement can be relative to outer shaft 120l, inner shaft 110, interventional device 190, stabilizing wire 160, and/or a portion of a body lumen. In some embodiments, while outer shaft 120 moves, inner shaft 110, interventional device 190, and/or stabilizing wire 160 can be stationary, moving in the same direction (e.g., at a same or different speed), or moving in a different (e.g., opposite) direction. In some embodiments, while inner shaft 110 moves, outer shaft 120 interventional device 190, and/or stabilizing wire 160 can be stationary, moving in the same direction (e.g., at a same or different speed), or moving in a different (e.g., opposite) direction. In some embodiments, while interventional device 190 and/or stabilizing wire 160 moves, outer shaft 120 and/or inner shaft 110 can be stationary, moving in the same direction (e.g., at a same or different speed), or moving in a different (e.g., opposite) direction.

As further shown in FIG. 10, outer shaft 120 has been retracted and interventional device 190 is unsheathed. In some embodiments, stabilizing wire 160 is movable with respect to outer shaft 120 and/or inner shaft 110 by independent control. Interventional device 190 can be unsheathed by retracting outer shaft 120 relative to stabilizing wire 160 and/or inner shaft 110. In some embodiments, stabilizing wire 160 is connected to inner shaft 110, and control of the position of interventional device 190 during and after retraction of outer shaft 120 can be achieved by control of inner shaft 110.

In some embodiments, filter 200 can accommodate passage of stabilizing wire and/or inner shaft 110 there through. For example, an opening in filter 200 can receive one or both of stabilizing wire 160 and inner shaft 110. By further example, different openings in filter 200 can be provided for each of stabilizing wire 160 and inner shaft 110. In some embodiments, stabilizing wire 160 and/or inner shaft 110 can pass on an outer side of filter 200.

Where an inflatable balloon (e.g., as shown in FIG. 2) is provided, such a balloon can be inflated to expand or further expand frame 191 and/or filter 200. For example, an interior region of the balloon can be fluidly connected, via inner shaft 110, to a port of connector 150. By providing a fluid, the balloon can be expanded, thereby expanding or further expanding frame 191 and/or filter 200.

Following one or more of the above-described operations, interventional device 190 can be maintained for any duration of time in an expanded state. For example, interventional device 190 can be maintained for a duration of time effective to provide therapeutic treatment (e.g., remodeling and/or drug delivery) to target anatomy, allow fluid flow through frame 191 and/or filter 200, and/or capture debris with filter 200. For example, flow 10 can be presented in the body lumen and in a vicinity of interventional device 190. Flow 10 can be in proximal direction to encounter an open end (e.g., distal end) of filter 200. Where flow 10 is in a direction of filter 200, filter 200 can capture debris that is above a certain size while optionally allowing perfusion of flow 10 through one or more pores and/or openings of filter 200.

Additionally or alternatively, interventional system 100 can be deployed at multiple locations. Interventional device 190 (e.g., including filter 200) can be collapsed by moving outer shaft 120 over interventional device 190. Interventional device 190 (e.g., including filter 200) can be moved to another target location, and one or more of the above-described operations can be repeated.

Additionally or alternatively, interventional system 100 can be removed. Interventional device 190 (e.g., including filter 200) can be collapsed by moving outer shaft 120 over interventional device 190. Components of interventional system 100 can be removed from the patient by retracting proximally (e.g., over a guidewire).

In some embodiments, while interventional device 190 (e.g., including filter 200) is collapsed by moving outer shaft 120 over interventional device 190, suction and/or aspiration can be provided to facilitate controlled capture and/or removal of any debris within filter 200. For example, a suction or aspiration device can be connected to connector 150 for applying a negative (e.g., relatively low) pressure at a distal end of outer shaft 120. Where filter 200 contains debris, such debris can be drawn into outer shaft 120 while filter 200 is collapsed therein. Such debris can be removed via outer shaft 120.

Additionally or alternatively, interventional device 190 (e.g., including filter 200) can be detached from inner shaft 110 and left as an implant within the patient. Following detachment, other components of interventional system 100 can be removed from the patient by retracting proximally (e.g., over a guidewire).

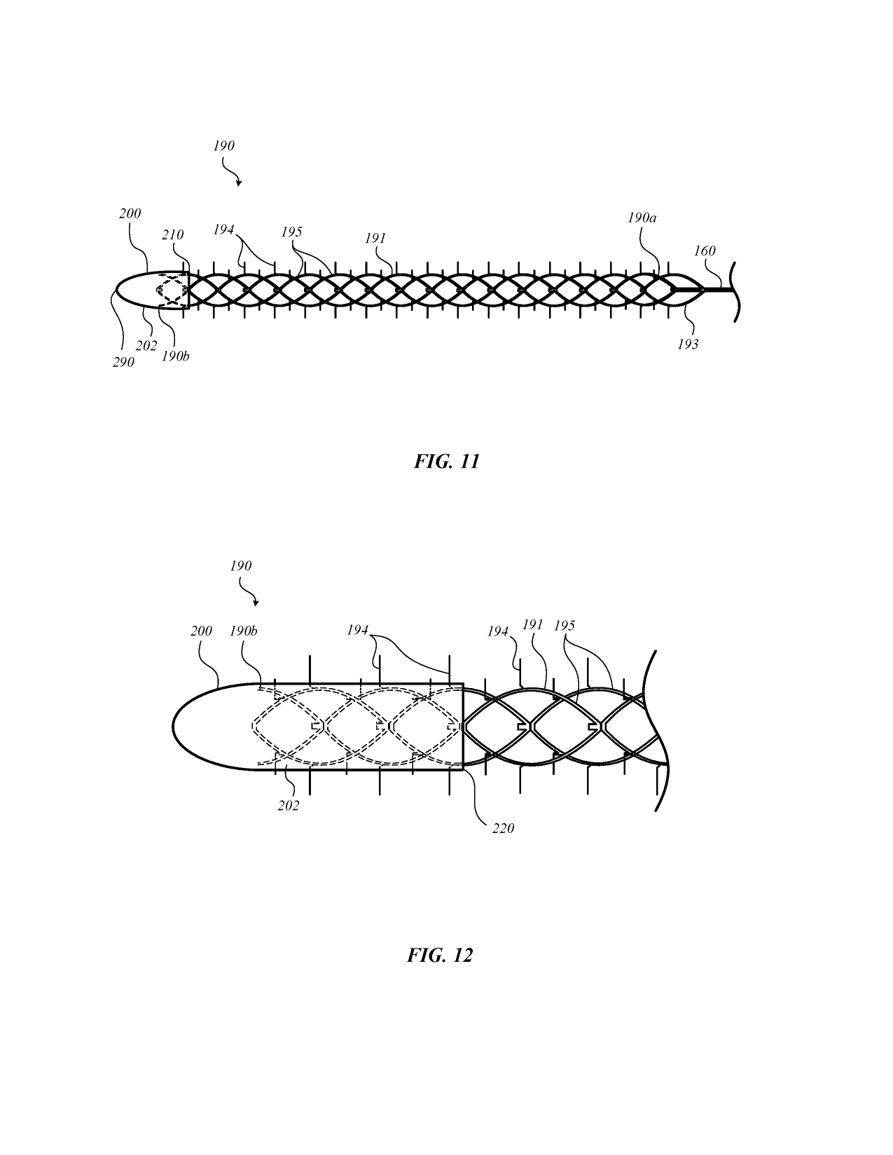

Referring now to FIGS. 11–18, an interventional device can include a filter covering a distal portion and/or end of the frame. Interventional device 190 of FIGS. 11–18 can include any one or more features described herein with respect to interventional device 190 of FIGS. 3–10. Filter 200 of FIGS. 11–18 can include any one or more features described herein with respect to filter 200 of FIGS. 3–10. While FIGS. 3–10 illustrate filter 200 on a proximal portion of interventional device 190 and FIGS. 11–18 illustrate filter 200 on a distal portion of interventional device 190, it will be understood that one or more filters can be positioned at any one or more locations with respect to frame 191 of interventional device 190. In some embodiments, interventional device 190 includes a filter covering a distal portion and/or end of frame 191 and a filter covering a proximal portion and/or end of frame 191.

As shown in FIG. 11, filter 200 can be provided at distal portion 190b of frame 191. For example, filter 200 can extend circumferentially and entirely about distal portion 190b of frame 191. Filter 200 can have a shape that generally conforms to and/or matches that of frame 191 at portions covered thereby. In some embodiments, filter 200 can have a maximum outer dimension that is greater than a maximum outer dimension of frame 191. As interventional device 190 expands, filter 200 can be urged radially outwardly along with radial expansion of frame 191. In some embodiments, filter 200 can constrain radial expansion of frame 191, for example where a maximum outer dimension of filter 200 is equal to or less than a maximum outer dimension of frame 191. Interventional device 190 of FIG. 11 can be implemented in any one of interventional systems 100 of FIGS. 1 and 2.

In some embodiments, as further shown in FIG. 11, filter 200 transitions from a first cross-sectional dimension at frame 191 to a second cross-sectional dimension at tapered end 290, wherein the first cross-sectional dimension is larger than the second cross-sectional dimension. For example, filter 200 can form open end 210, which can define a proximalmost extent of filter 200. Open end 210 can further define the first cross-sectional dimension, which can be a maximum cross-sectional dimension of filter 200. For example, an inner surface of wall 202 of filter 200 can abut and/or be close to an outer periphery of frame 191. In some embodiments, an inner dimension of wall 202 of filter 200 is equal to or approximately equal to an outer dimension of frame 191. In some embodiments, wall 202 of filter 200 constrains frame 191 while interventional device 190 is in an expanded state. From the first (e.g., maximum) cross-sectional dimension, filter 200 tapers to the second cross-sectional dimension, for example distal to distal portion 190b of frame 191. Accordingly, filter 200 can define an opening at open end 210 for receiving debris therein, and filter 200 can further capture such debris to be surrounded by wall 202 and blocked by a closure formed at a portion of filter 200 at tapered end 290.

Referring now to FIG. 12, a filter can be provided in an engaged arrangement with respect to a frame of an interventional device with one or more protruding features. As shown in FIG. 12, filter 200 can extend across a portion of frame 191 that supports one or more protruding features 194. In some embodiments, as protruding features 194 extend radially outwardly from frame 191 and as filter 200 is provided in close proximity to frame 191, one or more protruding features 194 positioned along the length of filter 200 extends through and beyond an outer surface of filter 200. In some embodiments, one or more protruding features 194 transition from a constrained state within filter 200 while interventional device 190 is in a collapsed state to an extended state protruding through wall 202 of filter 200 while interventional device 190 is in an expanded state. In some embodiments, while one or more protruding features 194 extend through wall 202 of filter 200, at least a portion of filter 200 is axially secured to frame 191. For example, filter 200 can be engaged by one or more protruding features 194 extending therethrough, and filter 200 can be maintained in a given axial alignment with respect to frame 191, such that at least a portion of filter 200 does not move axially with respect to frame 191. It will be understood that while some portions (e.g., a proximal portion) of filter 200 can be secured, other portions (e.g., a distal portion) of filter 200 can be permitted to move, such as portions not engaged by and/or between one or more protruding features 194. In some embodiments, one or more protruding features 194 of interventional device 190 are positioned to be axially offset with respect to filter 200. For example, at least a portion (e.g., distal portion 190b) of frame 191 can be covered by filter 200, at least a portion (e.g., proximal portion 190a) of frame 191 can be uncovered by filter 200, and one or more protruding features 194 of interventional device 190 can extend radially outwardly without extending through filter 200. In some embodiments, every one of protruding features 194 of interventional device 190 is positioned to extend through filter 200. Interventional device 190 of FIG. 12 can be implemented in any one of interventional systems 100 of FIGS. 1 and 2 and/or configured to include one or more of the features described herein with respect to interventional device 190 of FIG. 11.

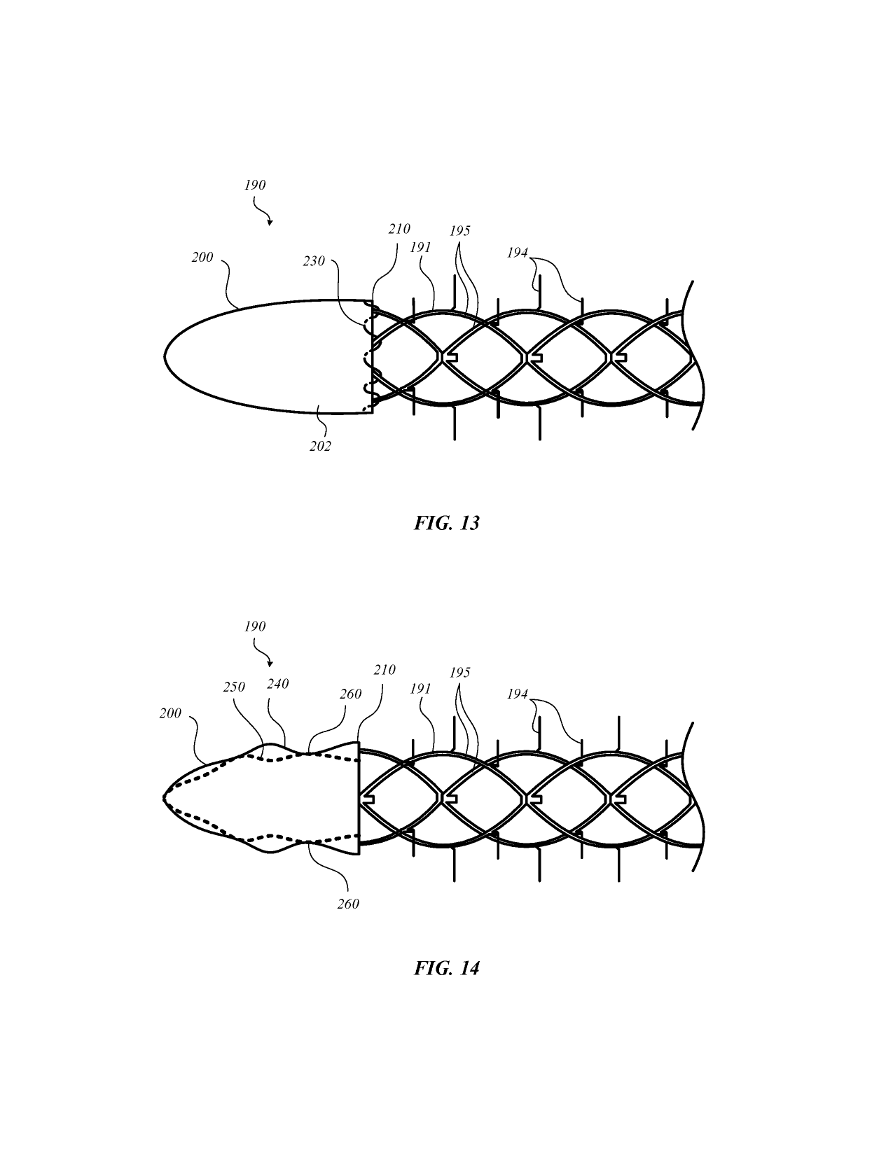

Referring now to FIG. 13, a filter can be provided in an engaged arrangement with respect to a frame of an interventional device with one or more strands. As shown in FIG. 13, filter 200 can extend across a portion of frame 191 that defines struts 195. In some embodiments, open end 210 of filter 200 is secured to frame 191 by one or more strands 230 that extend through at least a portion of wall 202 and about one or more of struts 195. For example, strand 230 can extend in an alternating pattern to extend through the wall 202 (e.g., at or near open end 210) and about an inner side of struts 195. Accordingly, strand 230 can provide tension that pulls filter 200 toward frame 191. In some embodiments, strand 230 extends about an entire circumference of frame 191 and/or open end 210. Strand 230 can optionally extend to an inner side of each strut 195 across which it extends. Between each strut 195, strand 230 can extend through and/or otherwise engage wall 202 of filter 200 at or near open end 210. In some embodiments, while strand 230 extends through wall 202 of filter 200 and about one or more struts 195, at least a portion (e.g., open end 210) of filter 200 is axially and/or radially secured to frame 191. For example, filter 200 can be engaged by one or more strands 230 extending therethrough, and filter 200 can be maintained in a given axial alignment with respect to frame 191, such that at least a portion (e.g., open end 210) of filter 200 does not move axially with respect to frame 191. It will be understood that while some portions (e.g., open end 210 and/or proximal portion) of filter 200 can be secured, other portions (e.g., a distal portion) of filter 200 can be permitted to move, such as portions not engaged by and/or between one or more strands 230. In some embodiments, at least a portion (e.g., distal portion 190b) of frame 191 can be covered by filter 200, at least a portion (e.g., proximal portion 190a) of frame 191 can be uncovered by filter 200, and one or more strands 230 can secure open end 210 there between. In some embodiments, one or more strands 230 secure open end 210 of filter 200 to a distal end of frame 191. Interventional device 190 of FIG. 13 can be implemented in any one of interventional systems 100 of FIGS. 1 and 2 and/or configured to include one or more of the features described herein with respect to interventional devices 190 of FIGS. 11 and 12. For example, open end 210 of filter 200 can be coupled to frame 191 by one or more strands 230 and/or one or more protruding features 194 of interventional device 190 can extend through wall 202 of filter 200.

Referring now to FIG. 14, a filter can be provided in an engaged arrangement with respect to a frame of an interventional device with coupled layers. As shown in FIG. 14, filter 200 can extend across a portion of frame 191 that defines struts 195. In some embodiments, filter 200 includes multiple layers, such as outer layer 240 and inner layer 250. In some embodiments, at least a portion of outer layer 240 extends across a radially outer surface of frame 191 (e.g., radially outer surfaces of struts 195), and at least a portion of inner layer 250 extends across a radially inner surface of frame 191 (e.g., radially inner surfaces of struts 195. For example, strand 230 can extend in an alternating pattern to extend through the wall 202 (e.g., at or near open end 210) and about an inner side of struts 195. In some embodiments, outer layer 240 and inner layer 250 are coupled to each other at one or more locations along a length of frame 191. For example, one or more couplings 260 can be provided at one or more interstices defined by struts 195. Such interstices can define openings that extend radially through frame 191. At such locations, outer layer 240 and inner layer 250 can be joined together by one or more couplings 260. In some embodiments, couplings 260 are formed by adhesion, fusion, and/or sintering of outer layer 240 and inner layer 250. In some embodiments, couplings 260 are formed by stitching, interweaving, and/or braiding of outer layer 240 and inner layer 250. Couplings 260 can be provided at various locations, including circumferentially and/or axially distributed across filter 200 and/or frame 191. While outer layer 240 and inner layer 250 can extend across radially opposite sides of corresponding struts 195, outer layer 240 and inner layer 250 can be joined together by couplings 260. As such, the locations of such couplings 260 with respect to frame 191 can be maintained to secure filter 200 to frame 191. In some embodiments, couplings 260 maintain at least a portion of filter 200 to be axially and/or radially secured to frame 191, such that at least a portion (e.g., proximal portion) of filter 200 does not move axially with respect to frame 191. It will be understood that while some portions (e.g., proximal portion) of filter 200 can be secured, other portions (e.g., a distal portion) of filter 200 can be permitted to move, such as portions not engaged by couplings 260 between struts 195. In some embodiments, at least a portion (e.g., distal portion 190b) of frame 191 can be covered by filter 200, at least a portion (e.g., proximal portion 190a) of frame 191 can be uncovered by filter 200. Interventional device 190 of FIG. 14 can be implemented in any one of interventional systems 100 of FIGS. 1 and 2 and/or configured to include one or more of the features described herein with respect to interventional devices 190 of FIGS. 11–13. For example, outer and inner layers 240 and 250 of filter 200 can be coupled together between some of struts 195 of frame 191, open end 210 of filter 200 can be coupled to frame 191 by one or more strands 230, and/or one or more protruding features 194 of interventional device 190 can extend through wall 202 (e.g., outer and inner layers 240 and 250) of filter 200.

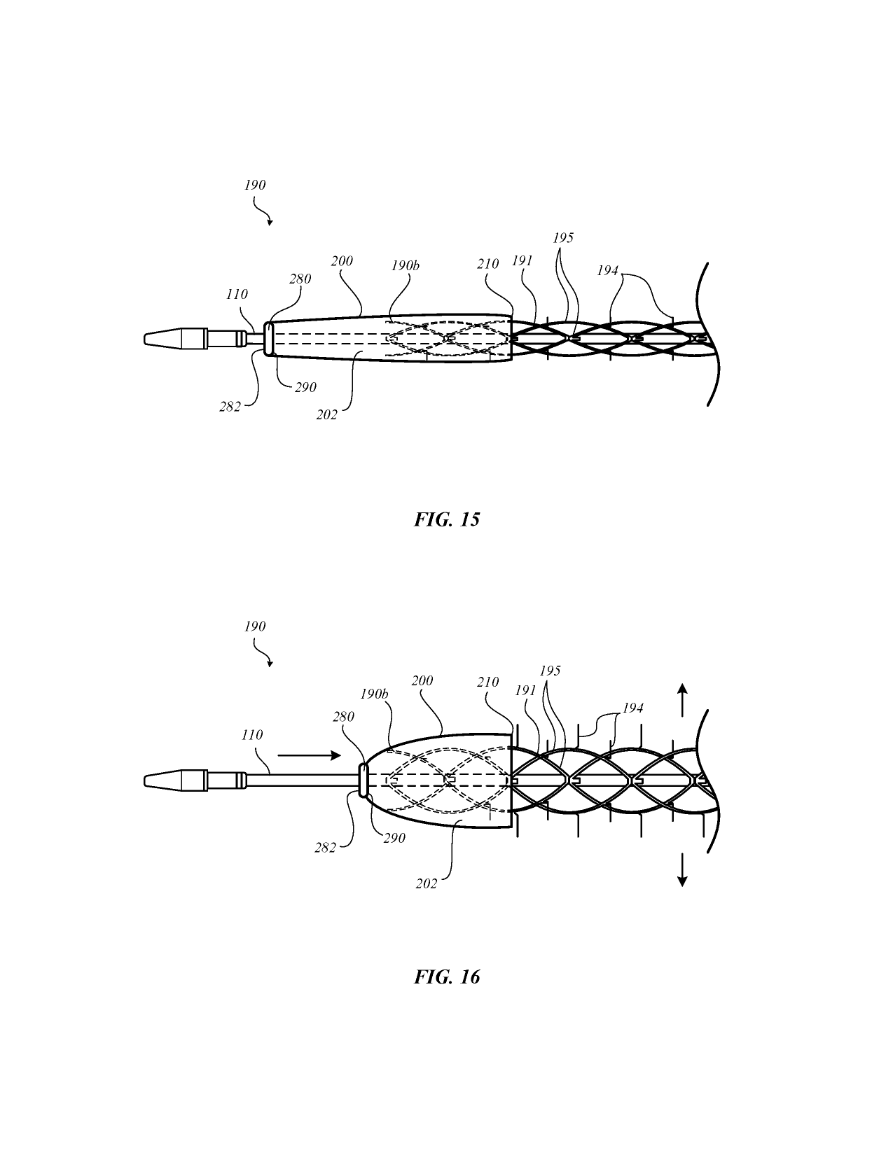

Referring now to FIGS. 15 and 16, an interventional device can be provided with features to facilitate expansion of a frame thereof with a filter thereon. Interventional device 190 of FIGS. 14 and 15 can be implemented in any one of interventional systems 100 of FIGS. 1 and 2 and/or configured to include one or more of the features described herein with respect to interventional devices 190 of FIGS. 11–16. For example, engagement between filter 200 and frame 191 can be provided with one or more protruding features 194 extending from frame 191 and through filter 200, open end 210 of filter 200 can be coupled to frame 191 by one or more strands, and/or outer and inner layers of filter 200 can be coupled together between some of struts 195 of frame 191. It will be understood that interventional device 190 can undergo changes to its size and/or shape when transitioning between a collapsed state (FIG. 15) and an expanded state (FIG. 16). Where filter 200 is coupled to frame 191 at or near open end 210 thereof, tapered end 290 of filter 200 can be configured to accommodate such changes while maintaining engagement at or near open end 210. In some embodiments, filter 200 includes collar 280 at or near tapered end 290 of filter 200. Collar 280 can provide opening 282 through which inner shaft 110 and/or another component can extend. Opening 282 can be configured (e.g., sized) to accommodate passage of inner shaft 110 and/or another component while also limiting passage of debris therethrough. In some embodiments, collar 280 can include a material and/or construction that is different from that of wall 202 of filter 200. For example, collar 280 can include an annular ring of a flexible material, such as a plastic, a polymer (e.g., silicon), and/or a combination thereof. Collar 280 can facilitate sliding along inner shaft 110. For example, while open end 210 or another proximal portion of filter 200 is secured to frame 191, tapered end 290 of filter 200 can be permitted to move (e.g. slide) to accommodate expansion of frame 191. In some embodiments, as shown in FIG. 16, frame 191 expands radially outwardly. Radial expansion of frame 191 can optionally include longitudinal foreshortening of frame 191. In some embodiments, as further shown in FIG. 16, radial expansion of frame 191 can also urge filter 200 to expand radially outwardly and/or foreshorten longitudinally. During expansion of frame 191, collar 280 can travel longitudinally (e.g., toward frame 191) to accommodate such transitions. Longitudinal travel of collar 280 in a proximal direction can optionally be limited by contact with frame 191 and/or another structure. In some embodiments, interventional device 190 can transition from the expanded state of FIG. 16 to the collapsed state of FIG. 15. Such a transition can include longitudinal motion (e.g., sliding) of collar 280 away from frame 191 (e.g., distally).

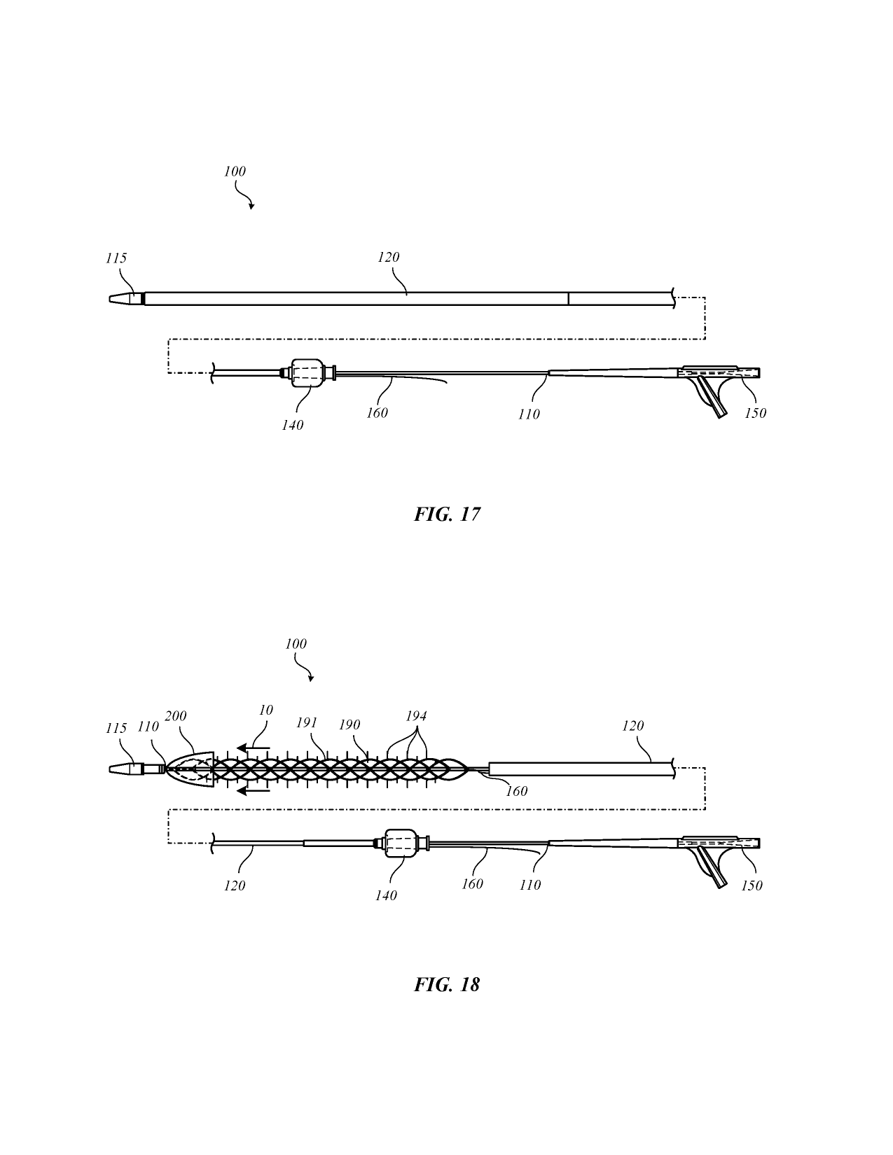

Referring now to FIGS. 17 and 18, an interventional device can be delivered to a target location for deployment thereat. Methods described herein provide delivery of interventional device 190 to a target delivery location by operation of interventional system 100. While methods in their various stages are discussed and illustrated herein, it will be understood that multiple variations of each method are also contemplated. For example, the methods can be performed in various orders of operations, with additional operations, or with fewer operations. Interventional system 100 of FIGS. 17 and 18 can be implemented as any one of interventional systems 100 of FIGS. 1 and 2, and interventional devices 190 of FIGS. 17 and 18 can be configured to include one or more of the features described herein with respect to interventional devices 190 of FIGS. 11-16.

As shown in FIG. 17, interventional system 100 is provided with outer shaft 120 covering or ensheathing other components of interventional system 100. For example, outer shaft 120 can extend to tip 115 positioned at a distal end of inner shaft 110. Inner shaft 110 can extend within outer shaft 120, with a length thereof accessible proximal to a proximal end of outer shaft 120 (e.g., at outer shaft hub 140). Additionally or alternatively, connector 150 can be accessible proximal to a proximal end of outer shaft 120 (e.g., at outer shaft hub 140). As discussed above, a guidewire can be advanced ahead of tip 115 (e.g., through inner shaft 110) to provide a pathway for advancement of other components of interventional system 100. In some embodiments, stabilization wire 160 is accessible on a proximal side of interventional system 100, such through outer shaft hub 140. For example, stabilization wire 160 can be connected to an interventional device within outer shaft 120.

As shown in FIG. 18, interventional system 100 can be provided with interventional device 190 that is positioned over inner shaft 110 (and/or an inflatable balloon, not shown in FIG. 18) for expansion and delivery of interventional device 190 to a target delivery location. By positioning interventional device 190 over and about inner shaft 110, interventional device 190 can be ready to be expanded immediately upon unsheathing with respect to outer shaft 120.

As further shown in FIG. 18, outer shaft 120 can be moved to unsheath interventional device 190 and other components of interventional system 100. For example, once the distal region of interventional system 100 is positioned at a desired location, outer shaft 120 is configured to be at least partially proximally retracted relative to inner shaft 110 by retracting outer shaft hub 140 relative to connector 150. Once outer shaft 120 is partially retracted, at least a portion of interventional device 190 and/or a balloon (where provided) is unsheathed. In some embodiments, frame 191, protruding features 194, and/or filter 200 of interventional device 190 are configured to radially expand outwardly away from inner shaft 110.

As used herein, movement of various components can be relative to other components of interventional system 100 and/or relative to a position apart from interventional system 100 (e.g., a position within the anatomy of the patient, target delivery location, and/or tissue). The directions “proximal” and “distal” can be with respect to interventional system 100, a component thereof, and/or a position apart from interventional system 100. For example, movement can be relative to outer shaft 120l, inner shaft 110, interventional device 190, stabilizing wire 160, and/or a portion of a body lumen. In some embodiments, while outer shaft 120 moves, inner shaft 110, interventional device 190, and/or stabilizing wire 160 can be stationary, moving in the same direction (e.g., at a same or different speed), or moving in a different (e.g., opposite) direction. In some embodiments, while inner shaft 110 moves, outer shaft 120 interventional device 190, and/or stabilizing wire 160 can be stationary, moving in the same direction (e.g., at a same or different speed), or moving in a different (e.g., opposite) direction. In some embodiments, while interventional device 190 and/or stabilizing wire 160 moves, outer shaft 120 and/or inner shaft 110 can be stationary, moving in the same direction (e.g., at a same or different speed), or moving in a different (e.g., opposite) direction.

As further shown in FIG. 18, outer shaft 120 has been retracted and interventional device 190 is unsheathed. In some embodiments, stabilizing wire 160 is movable with respect to outer shaft 120 and/or inner shaft 110 by independent control. Interventional device 190 can be unsheathed by retracting outer shaft 120 relative to stabilizing wire 160 and/or inner shaft 110. In some embodiments, stabilizing wire 160 is connected to inner shaft 110, and control of the position of interventional device 190 during and after retraction of outer shaft 120 can be achieved by control of inner shaft 110.

In some embodiments, filter 200 can accommodate passage of inner shaft 110 there through. For example, an opening in filter 200 can receive inner shaft 110. In some embodiments, inner shaft 110 can pass on an outer side of filter 200 (e.g., with or without passing through at least a portion of frame 191).

Where an inflatable balloon (e.g., as shown in FIG. 2) is provided, such a balloon can be inflated to expand or further expand frame 191 and/or filter 200. For example, an interior region of the balloon can be fluidly connected, via inner shaft 110, to a port of connector 150. By providing a fluid, the balloon can be expanded, thereby expanding or further expanding frame 191 and/or filter 200.

Following one or more of the above-described operations, interventional device 190 can be maintained for any duration of time in an expanded state. For example, interventional device 190 can be maintained for a duration of time effective to provide therapeutic treatment (e.g., remodeling and/or drug delivery) to target anatomy, allow fluid flow through frame 191 and/or filter 200, and/or capture debris with filter 200. For example, flow 10 can be presented in the body lumen and in a vicinity of interventional device 190. Flow 10 can be in distal direction to encounter the open end (e.g., proximal end) of filter 200. Where flow 10 is in a direction of filter 200, filter 200 can capture debris that is above a certain size while optionally allowing perfusion of flow 10 through one or more pores and/or openings of filter 200.

Additionally or alternatively, interventional system 100 can be deployed at multiple locations. Interventional device 190 (e.g., including filter 200) can be collapsed by moving outer shaft 120 over interventional device 190. Interventional device 190 (e.g., including filter 200) can be moved to another target location, and one or more of the above-described operations can be repeated.

Additionally or alternatively, interventional system 100 can be removed. Interventional device 190 (e.g., including filter 200) can be collapsed by moving outer shaft 120 over interventional device 190. Components of interventional system 100 can be removed from the patient by retracting proximally (e.g., over a guidewire).

In some embodiments, while interventional device 190 (e.g., including filter 200) is collapsed by moving outer shaft 120 over interventional device 190, suction and/or aspiration can be provided to facilitate controlled capture and/or removal of any debris within filter 200. For example, a suction or aspiration device can be connected to connector 150 for applying a negative (e.g., relatively low) pressure at a distal end of outer shaft 120. Where filter 200 contains debris, such debris can be drawn into outer shaft 120 while filter 200 is collapsed therein. Such debris can be removed via outer shaft 120.

Additionally or alternatively, interventional device 190 (e.g., including filter 200) can be detached from inner shaft 110 and left as an implant within the patient. Following detachment, other components of interventional system 100 can be removed from the patient by retracting proximally (e.g., over a guidewire).

While the interventional devices described herein have the features shown, it will be understood that a variety of different stents and other devices can be used with the interventional systems described herein. Various features are set forth below by way of example, and not by limitation.

Regarding such interventional devices and other devices, the material(s) for forming the frame, struts, and/or protruding features described herein can be selected based on mechanical and/or thermal properties, such as strength, ductility, hardness, elasticity, flexibility, flexural modulus, flexural strength, plasticity, stiffness, emissivity, thermal conductivity, specific heat, thermal diffusivity, thermal expansion, any of a variety of other properties, or a combination thereof. If formed from a material having thermal properties, the material can be activated to deliver thermal treatment to the desired treatment site. Regardless of the material, the frame, struts, and/or protruding features can be formed from a tube or a wire, such as a solid wire, by laser cutting or other suitable techniques. When formed from the wire, a portion of the wire can be removed by chemical etching or another suitable method to create an inner dimension of the interventional device.

Interventional devices (e.g., the frame and the struts) can be sized and shaped for placement within various body lumens, including blood vessels, while not rupturing the vessel. For example, several interventional devices and other structures can have radial strength that allows for features of the body lumen (e.g., vessel wall) to receive drugs without dissection or damage thereto. Vessels in which the interventional devices described herein may be sized and shaped for placement include arteries, such as coronary arteries, peripheral arteries, carotid arteries, circle of willis, anterior cerebral artery, middle cerebral artery, posterior cerebral artery, any of the lenticulostriate arteries, renal arteries, femoral arteries, veins, such as cerebral veins, saphenous veins, arteriovenous fistulas, or any other vessel that may contain a treatment site. Interventional devices can have a variety of shapes, including a cube, a rectangular prism, a cylinder, a cone, a pyramid, or variations thereof.

Interventional devices and other structures having protruding features can include a variety of dimensions (in both the low-profile delivery state and expanded deployed state). These embodiments can provide for expansion that enables usage in a variety of situations covering a wide range of dimensions, such as to treat and/or prevent dissection. Regardless of the shape, interventional devices can have a length of about 0.25 mm, about 0.5 mm, about 1 mm, about 2 mm, about 3 mm, about 4 mm, about 5 mm, about 6 mm, about 7 mm, about 8 mm, about 9 mm, about 10 mm, about 12 mm, about 14 mm, about 16 mm, about 18 mm, about 20 mm, about 30 mm, about 40 mm, about 50 mm, about 60 mm, about 70 mm, about 80 mm, about 90 mm, or about 100 mm. In addition, an interventional device shaped into a cube, a rectangular prism, or a pyramid can have a width of about 0.25 mm, about 0.5 mm, about 1 mm, about 2 mm, about 3 mm, about 4 mm, about 5 mm, about 6 mm, about 7 mm, about 8 mm, about 9 mm, about 10 mm, about 12 mm, about 14 mm, about 16 mm, about 18 mm, about 20 mm, about 25 mm, or about 30 mm. Moreover, an interventional device shaped into a cylinder or a cone can have a diameter of about 0.25 mm, about 0.5 mm, about 1 mm, about 2 mm, about 3 mm, about 4 mm, about 5 mm, about 6 mm, about 7 mm, about 8 mm, about 9 mm, about 10 mm, about 12 mm, about 14 mm, about 16 mm, about 18 mm, about 20 mm, about 25 mm, about 30 mm, about 35 mm, about 40 mm, or about 50 mm. The width or the diameter of the interventional device can decrementally decrease along a length of the interventional device. In addition, the interventional device can be sized and shaped to prepare the body lumen for certain procedures, such as an interventional device placement procedure.

An interventional device and/or other expandable structures in the expanded state can have a cross-sectional dimension of about 2 mm to about 10 mm, inclusive of the expanded protruding features. For example, a frame can have a cross-sectional dimension of about 1 mm to about 9 mm and the protruding features can each have a length from about 0.1 mm to about 1.5 mm. In some embodiments, the interventional device has an overall cross-sectional dimension of about 4 mm with the frame having a cross-sectional dimension of about 2 mm and the protruding features each having a length of about 1 mm. In some embodiments, the interventional device has an overall cross-sectional dimension of about 6 mm with the frame having a cross-sectional dimension of about 4 mm and the protruding features each having a length of about 1 mm. In further embodiments, the protruding features can have a plurality of lengths such that the length of the protruding features of an interventional device or other expandable structure differs. For example, an interventional device can include protruding features having a length of about 0.2 mm, about 0.5 mm, and about 1 mm.

Profiles of the interventional devices or other structures can be sized such that the interventional devices or other structures are compatible with a wide range of catheter sizes. Embodiments in accordance with the present technology can include interventional devices or other structures designed to receive a guidewire, such as guidewires having a diameter of 0.010, 0.014, 0.018, 0.035, or 0.038 inch. In several embodiments, the interventional device or scaffold structure can be sized and designed for delivery via a micro-catheter that it is pushed through. In some embodiments, interventional devices or structures can be incorporated into an interventional system, including modular or single unit interventional systems.

Interventional devices and other structures described herein can include a marking for visualization of the interventional device within the body lumen, such as one or more radiopaque markers. The radiopaque markers can be formed from Clearfil Photo Core PLT®, tantalum, titanium, tungsten, barium sulfate, and zirconium oxide, or another suitable radiopaque marking. The markings can be formed on a proximal portion of the interventional device, a distal portion, an intermediate portion, or a combination thereof. The markings can be a band, a coil, a clip, filled into one or more portions of a tube in the interventional device, plated onto one or more portions of the interventional device, or a combination thereof. Regardless of the type of marking, the marking can be coined, swaged, wrapped, or encased along, or onto any portion of the interventional device.

Interventional devices and other structures can be flexible enough to track through various anatomical features, including those having a curvature. The flexible properties of the interventional device and other structures can be provided by the material from they are formed. In addition, flexible properties can also be provided by fracturing one or more of the members engaging with and extending between two or more rows of struts. Additionally, the interventional device or other structure can be readily deployed and expanded, and retracted and contracted. The interventional device or other structure can also be readily repositioned within a vessel or other body lumen.

In several embodiments, a drug-eluting compound is coated onto at least a portion of the protruding features, the frame, the struts, and/or the balloon. The coating can be any suitable coating known to one of ordinary skill in the art suitable to deliver the drug to the wall. For example, suitable coatings include, but are not limited to a snow coating or a crystalline coating having edges configured to remain in the wall. The drug-eluting compound can be a synthetic or biological polymer coated into a variety of different patterns and thicknesses suitable for delivering the drug contained therein. In other embodiments, the protruding features themselves may be composed of drug-eluting materials. The drug carried by the drug-eluting compound and/or the protruding features in accordance with the present technology can be any drug suitable for treating the treatment site in which the interventional device will be placed and may or may not include an excipient. For example, the drug can be an anti-proliferative, an anti-neoplastic, a migration inhibitor, an enhanced healing factor, an immunosuppressive, an anti-thrombotic, a blood thinner, or a radioactive compound. Examples of anti-neoplastics include, but are not limited to, siroliums, tacrolimus, everolimus, leflunomide, M-prednisolone, dexamethasone, cyclosporine, mycophenolic acid, mizoribine, interferon, and tranilast. Examples of anti-proliferatives include, but are not limited to, taxol/paclitaxel, actinomycin, methotrexate, angiopeptin, vincristine, mitmycine, statins, c-myc antisense, Abbot ABT-578, RestinASE, 2-chloro-deoxyadenosine, and PCNA ribozyme. Examples of migration inhibitors, but are not limited to, include batimistat, prolyl hydrosylase, halofunginone, c-preteinase inhibitors, and probucol. Examples of enhanced healing factors include, but are not limited to, BCP 671, VEGF, estradiols, NO donor comounds, and EPC antibodies. Examples, of radioactive compounds include, but are not limited to, strontium-89 chloride (Metastron®), samarium-153 (Quadramet®), radium-223 dichloride (Xofigo®), yttrium-90, and iodine-131. In some embodiments, the drug-eluting compound and/or the protruding features can carry more than one drug.

In some embodiments, the protruding features can include textured (e.g., ribbed) surfaces which is expected to provide greater surface area for drug-delivery. Moreover, any protruding features can include a textured surface such as a ribbed surface (vertical, horizontal, radial, or circular relative to a longitudinal plane of the protruding feature), a cross-hatched surface, an isotropic surface, or other surface types suitable for providing greater surface area for drug-delivery.

The protruding features can be sized and shaped to engage with and/or penetrate an occlusion, a neointima, an intima, an internal elastic lamina (IEL) a media, an external elastic lamina (EEL), an adventitia, or a combination thereof. The protruding features can also be sized and shaped to engage with and/or penetrate a tissue and/or structure adjacent to the body lumen in which the interventional device is to be placed while not rupturing the body lumen. For example, the interventional device can include square protruding features sized and configured to penetrate into the intima and/or the media of a body lumen, pointed protruding features sized and configured to penetrate and extend into the media, and/or the IEL. In addition, protruding features can be configured to bend in one or more directions relative to a longitudinal axis of the interventional device to engage with and/or penetrate a portion of the body lumen described herein. In several embodiments, the protruding features can penetrate deeper into the wall of a diseased body lumen, such as a vessel, compared to an interventional device lacking protruding features. In addition, the interventional device can allow for blood to flow even while in the expanded position and with drug-eluting on-going.

Various protruding features described herein can deliver drugs deeper into a vessel wall than possible via angioplasty balloons or other existing devices. In addition to carrying one or more drugs for treatment of the site, the protruding features can also carry a molecule suitable for degrading a portion of the occlusion, neointima, and/or intima to allow the protruding features to penetrate deeper in to the vessel wall than without the molecule. For example, the molecule suitable for degradation can be an enzyme, such as elastase, collagenase, or a proteinase, such as, metalloproteinases, serine proteinases, cysteine proteinases, extracellular sulfatases, hyaluronidases, lysyl oxidases, lysyl hydroxylases, or a combination thereof.

Further, it will also be appreciated that interventional devices can carry one or more protruding features on one or more portions of the interventional device. For example, the interventional devices can carry about 5 protruding features, about 10 protruding features, about 15 protruding features, about 20 protruding features, about 30 protruding features, about 40 protruding features, about 50 protruding features, about 60 protruding features, about 70 protruding features, about 80 protruding features, about 90 protruding features, or about 100 protruding features. The protruding features can be carried by the frame, the struts, or a combination thereof. The number of protruding features can vary depending upon, for example, the target treatment site, the type of drug being delivered, and size of the interventional device, etc. In addition, the protruding features carried by the interventional device can be different types of the protruding features disclosed herein.

In some embodiments, once positioned against a body lumen wall (e.g., a vessel wall), tissue and/or fluid can interact with the protruding feature to dissolve the drug and selectively release it from the reservoir. In other embodiments, the protruding feature can be configured to deliver the drug via a variety of means once the interventional device is expanded. Protruding features are accordingly expected to provide an effective means for selectively delivering a drug to a desired location, while reducing inadvertent loss or release of drugs. In other embodiments, the interventional device can include more than one protruding feature, or a protruding feature having more than one reservoir. In several embodiments, the interventional device including protruding features can have the protruding feature, such as the coating or the reservoir, concealed (e.g., recessed) until the interventional device is positioned at the treatment site. Once positioned at the target site, the protruding feature can be revealed (e.g., expanded/projected, etc.) during and/or after expansion of the interventional device. This is expected to reduce any loss of the drug carried by the protruding feature during delivery to the treatment site.

In some embodiments, the interventional devices can further include a material (e.g., PTFE, Dacron, polyamides, such as nylon and/or polyurethane based materials, silicone, etc.) positioned over an interventional device, scaffold or other structure having protruding features covering at least a portion of the outer surface area. In some embodiments, the material covers the entire outer surface area. The material can be a mesh or a braid. In some embodiments, the material can be configured to increase a surface area of the interventional device useful for providing additional surface area of the interventional device for coating with a drug. In other embodiments, the material can further be configured to allow blood flow through the inner diameter of the interventional device and/or limit blood flow to an outer dimension of the interventional device. In additional embodiments, the material can create a barrier between fluid flow (e.g., blood flow) and the drug-delivery locations. In addition, the material can be configured to prevent debris from the wall of the body lumen from entering the bloodstream. In such embodiments, the associated systems and devices can be used for temporary dissection tacking or coverage of a region that may have been perforated during a procedure.

The embodiments described herein provide interventional systems for one or more structures having a means for delivering drugs to a specific region within a body lumen, such as the vasculature, while still allowing fluid (e.g., blood) to flow through the treatment area where the structure has been placed and/or other devices or treatment means within the adjacent body lumen. In some embodiments, the fluid is temporary prevented from flowing through the treatment area while one or more regions of systems is delivered, deployed, positioned, and/or removed from the body lumen. In addition, the interventional systems can be configured to prepare the body lumen for treatment, by raking the interventional device, pulling the interventional device, turning the interventional device, or a combination thereof, proximal or distal to the treatment site. In other embodiments, the interventional systems can be configured to rotate the interventional device when mechanical force is applied.

The systems disclosed herein can provide for adjustment, recapture, and/or redeployment of the associated interventional devices or other structures, and/or deployment of a different interventional device or other structure, allowing a practitioner to more effectively to treat a desired region more accurately and deliberately. In several embodiments, the interventional device or other delivery structure can be deployed for a temporary period (e.g., for less than 24 hours), and then retracted and removed. In these embodiments, the protruding features can engage with and/or pierce the lumen wall and remain therein after the interventional device or other delivery structure is removed or can be retracted and removed with the interventional device or other delivery structure. The interventional device can be configured to self-expand, or partially self-expand, when deployed from the interventional system and also be configured to further expand within the body lumen when the balloon is expanded therein. The interventional device can also be configured to post-dilate when removed from the body lumen. In other embodiments, the interventional device or other delivery structure can be deployed for a long-term temporary period (e.g., for less than 2 weeks, less than one month, less than 6 months, less than one year), and then retracted and removed. In some embodiments, a different interventional device or delivery structure can be deployed after a first interventional device or delivery structure has been retraced and removed. The duration of deployment and duration after removal before deployment of the different interventional device or delivery structure can vary from minutes, to hours, to days, to weeks, to months, or to years. In these embodiments, removal of the first interventional device or delivery structure and deployment of a different interventional device or delivery structure can occur once, twice, three times, four times, five times, six times, seven times, eight times, nine times, or ten times. Moreover, the embodiments described herein can allow for a lower profile system than currently available systems.

In the embodiments described herein and other embodiments configured in accordance with the present technology, interventional devices and other expandable structures may include non-protruding features, such as deployable and/or expandable features, that are not configured for delivering a drug to a target location. For example, interventional devices and other expandable structures configured in accordance with the present technology can include one or more protruding features, one or more non-protruding features, or combinations thereof.

While many embodiments of the interventional devices and/or structures described herein include interventional devices, additional embodiments of the expandable elements, such as interventional devices and/or structures, can include non-drug-eluting interventional devices and/or non-drug-eluting structures. In these embodiments, the non-drug-eluting interventional devices may include one or more protruding members, such as spikes. The spikes can be configured to engage with and/or penetrate a portion of the body lumen or vessel. For example, the spikes can penetrate the vessel wall, thereby reducing and/or eliminating an elasticity of the vessel wall. In these embodiments, the protruding members can be configured to prevent the vessel wall from progressing inward toward the body lumen and restricting and/or constricting flow therein. The protruding members can be integrally formed with the struts, or disposed on the surface of the struts, extending radially outward from the struts toward the target tissue.

Various examples of aspects of the disclosure are described below as clauses for convenience. These are provided as examples, and do not limit the subject technology.

Clause A: an interventional device comprising: a frame comprising struts interconnected to each other and configured to expand from a collapsed configuration to an expanded configuration; protruding features extending radially outwardly from the frame when the frame is in the expanded configuration; an elongate member extending to the frame; and a filter having an open end and a tapered end, the open end being coupled to an end portion of the frame and circumferentially surrounding the end portion of the frame, wherein the elongate member extends through the tapered end of the filter.

Clause B: an interventional system comprising: an outer shaft; an elongate member ; and an interventional device comprising: a frame comprising struts interconnected to each other and configured to expand from a collapsed configuration to an expanded configuration, the frame surrounding a first portion of the elongate member; and a filter forming a mesh, circumferentially surrounding an end portion of the frame and a second portion of the elongate member, and extending longitudinally to the elongate member.

Clause C: a method comprising: positioning at least a portion of an interventional system within a body lumen, the interventional system including an interventional device in a collapsed configuration within an outer shaft of the interventional system; expanding a frame and a filter of the interventional device outside of the outer shaft, the frame and the filter surrounding a portion of an elongate member that extends through the outer shaft; capturing debris within the filter of the interventional device; and retracting the interventional device into the outer shaft.

One or more of the above clauses can include one or more of the features described below. It is noted that any of the following clauses can be combined in any combination with each other, and placed into a respective independent clause, e.g., clause A, B, or C.

Clause 1: the filter transitions from a first cross-sectional dimension at the open end to a second cross-sectional dimension at the tapered end, the first cross-sectional dimension being larger than the second cross-sectional dimension.

Clause 2: at least some of the protruding features extend through the filter.

Clause 3: the open end is coupled to the frame by a strand that extends through the filter and about some of the struts of the frame.

Clause 4: the filter comprises an outer layer about the frame and an inner layer within the frame, wherein the outer layer and the inner layer are coupled together between some of the struts of the frame.

Clause 5: the open end is coupled to the end portion of the frame, wherein the interventional device further comprises a collar extending annularly about the elongate member, wherein the collar is configured to slide along the elongate member.

Clause 6: the filter comprises a fabric.

Clause 7: the filter defines pores having a size of 40–150 microns.

Clause 8: the protruding features extend both radially outwardly and longitudinally away from the end portion of the frame.

Clause 9: the protruding features are monolithically formed with the frame.

Clause 10: an inflatable balloon on the elongate member and within the frame.

Clause 11: the interventional device further comprises protruding features extending radially outwardly from the frame when the frame is in the expanded configuration.

Clause 12: the elongate member is a stabilizing wire extending from the end portion of the frame and through the filter.

Clause 13: the interventional device further comprises one or more protruding features extending from the frame and through the filter.

Clause 14: the filter comprises an open end that is coupled to the frame by a strand that extends through the filter and about some of the struts of the frame.

Clause 15: expanding the interventional device comprises inflating a balloon positioned along the elongate member.

Clause 16: expanding the interventional device comprises extending protruding features of the interventional device from the frame and through the filter.

Clause 17: while expanded within the body lumen, a mesh of the filter provides selective passage there through.

A reference to an element in the singular is not intended to mean one and only one unless specifically so stated, but rather one or more. For example, “a” module may refer to one or more modules. An element proceeded by “a,” “an,” “the,” or “said” does not, without further constraints, preclude the existence of additional same elements.

Headings and subheadings, if any, are used for convenience only and do not limit the invention. The word exemplary is used to mean serving as an example or illustration. To the extent that the term include, have, or the like is used, such term is intended to be inclusive in a manner similar to the term comprise as comprise is interpreted when employed as a transitional word in a claim. Relational terms such as first and second and the like may be used to distinguish one entity or action from another without necessarily requiring or implying any actual such relationship or order between such entities or actions.

Phrases such as an aspect, the aspect, another aspect, some aspects, one or more aspects, an implementation, the implementation, another implementation, some implementations, one or more implementations, an embodiment, the embodiment, another embodiment, some embodiments, one or more embodiments, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

A phrase “at least one of” preceding a series of items, with the terms “and” or “or” to separate any of the items, modifies the list as a whole, rather than each member of the list. The phrase “at least one of” does not require selection of at least one item; rather, the phrase allows a meaning that includes at least one of any one of the items, and/or at least one of any combination of the items, and/or at least one of each of the items. By way of example, each of the phrases “at least one of A, B, and C” or “at least one of A, B, or C” refers to only A, only B, or only C; any combination of A, B, and C; and/or at least one of each of A, B, and C.

It is understood that the specific order or hierarchy of steps, operations, or processes disclosed is an illustration of exemplary approaches. Unless explicitly stated otherwise, it is understood that the specific order or hierarchy of steps, operations, or processes may be performed in different order. Some of the steps, operations, or processes may be performed simultaneously. The accompanying method claims, if any, present elements of the various steps, operations or processes in a sample order, and are not meant to be limited to the specific order or hierarchy presented. These may be performed in serial, linearly, in parallel or in different order. It should be understood that the described instructions, operations, and systems can generally be integrated together in a single software/hardware product or packaged into multiple software/hardware products.

In one aspect, a term coupled or the like may refer to being directly coupled. In another aspect, a term coupled or the like may refer to being indirectly coupled.

Terms such as top, bottom, front, rear, side, horizontal, vertical, and the like refer to an arbitrary frame of reference, rather than to the ordinary gravitational frame of reference. Thus, such a term may extend upwardly, downwardly, diagonally, or horizontally in a gravitational frame of reference.

The disclosure is provided to enable any person skilled in the art to practice the various aspects described herein. In some instances, well-known structures and components are shown in block diagram form in order to avoid obscuring the concepts of the subject technology. The disclosure provides various examples of the subject technology, and the subject technology is not limited to these examples. Various modifications to these aspects will be readily apparent to those skilled in the art, and the principles described herein may be applied to other aspects.

All structural and functional equivalents to the elements of the various aspects described throughout the disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112, sixth paragraph, unless the element is expressly recited using the phrase “means for” or, in the case of a method claim, the element is recited using the phrase “step for”.

The title, background, brief description of the drawings, abstract, and drawings are hereby incorporated into the disclosure and are provided as illustrative examples of the disclosure, not as restrictive descriptions. It is submitted with the understanding that they will not be used to limit the scope or meaning of the claims. In addition, in the detailed description, it can be seen that the description provides illustrative examples and the various features are grouped together in various implementations for the purpose of streamlining the disclosure. The method of disclosure is not to be interpreted as reflecting an intention that the claimed subject matter requires more features than are expressly recited in each claim. Rather, as the claims reflect, inventive subject matter lies in less than all features of a single disclosed configuration or operation. The claims are hereby incorporated into the detailed description, with each claim standing on its own as a separately claimed subject matter.

The claims are not intended to be limited to the aspects described herein, but are to be accorded the full scope consistent with the language of the claims and to encompass all legal equivalents. Notwithstanding, none of the claims are intended to embrace subject matter that fails to satisfy the requirements of the applicable patent law, nor should they be interpreted in such a way.

What is claimed is:

1. An interventional device comprising:
a frame comprising struts interconnected to each other and configured to expand from a collapsed configuration to an expanded configuration;
protruding features extending radially outwardly from the frame when the frame is in the expanded configuration;
an elongate member extending to the frame; and
a filter having an open end and a tapered end, the open end being coupled to an end portion of the frame and circumferentially surrounding the end portion of the frame, wherein the elongate member extends through the tapered end of the filter.

2. The interventional device of claim 1, wherein the filter transitions from a first cross-sectional dimension at the open end to a second cross-sectional dimension at the tapered end, the first cross-sectional dimension being larger than the second cross-sectional dimension.

3. The interventional device of claim 1, wherein at least some of the protruding features extend through the filter.

4. The interventional device of claim 1, wherein the open end is coupled to the frame by a strand that extends through the filter and about some of the struts of the frame.

5. The interventional device of claim 1, wherein the filter comprises an outer layer about the frame and an inner layer within the frame, wherein the outer layer and the inner layer are coupled together between some of the struts of the frame.

6. The interventional device of claim 1, wherein the open end is coupled to the end portion of the frame, wherein the interventional device further comprises a collar extending annularly about the elongate member, wherein the collar is configured to slide along the elongate member.

7. The interventional device of claim 1, wherein the filter comprises a fabric.

8. The interventional device of claim 1, wherein the filter defines pores having a size of 40–150 microns.

9. The interventional device of claim 1, wherein the protruding features extend both radially outwardly and longitudinally away from the end portion of the frame.

10. The interventional device of claim 1, wherein the protruding features are monolithically formed with the frame.

11. An interventional system comprising:
an outer shaft;
an elongate member ; and
an interventional device comprising:
a frame comprising struts interconnected to each other and configured to expand from a collapsed configuration to an expanded configuration, the frame surrounding a first portion of the elongate member; and
a filter forming a mesh, circumferentially surrounding an end portion of the frame and a second portion of the elongate member, and extending longitudinally to the elongate member.

12. The interventional system of claim 11, further comprising an inflatable balloon on the elongate member and within the frame.

13. The interventional system of claim 11, wherein the interventional device further comprises protruding features extending radially outwardly from the frame when the frame is in the expanded configuration.

14. The interventional system of claim 11, wherein the elongate member is a stabilizing wire extending from the end portion of the frame and through the filter.

15. The interventional system of claim 11, wherein the interventional device further comprises one or more protruding features extending from the frame and through the filter.

16. The interventional system of claim 11, wherein the filter comprises an open end that is coupled to the frame by a strand that extends through the filter and about some of the struts of the frame.

17. A method comprising:
positioning at least a portion of an interventional system within a body lumen, the interventional system including an interventional device in a collapsed configuration within an outer shaft of the interventional system;
expanding a frame and a filter of the interventional device outside of the outer shaft, the frame and the filter surrounding a portion of an elongate member that extends through the outer shaft;
capturing debris within the filter of the interventional device; and
retracting the interventional device into the outer shaft.

18. The method of claim 17, wherein expanding the interventional device comprises inflating a balloon positioned along the elongate member.

19. The method of claim 17, wherein expanding the interventional device comprises extending protruding features of the interventional device from the frame and through the filter.

20. The method of claim 17, wherein, while expanded within the body lumen, a mesh of the filter provides selective passage there through.
